(19) United States
(12) Patent Application Publication
(10) Pub. No.: US 2010/0152156 A1
(43) Pub. Date: Jun. 17, 2010

(54) 2-Azetidinone Derivatives For The Treatment Of Hyperlipidaemic Diseases

(75) Inventors: Mikael Dahlstrom, Molndal (SE); Fana Hunegnaw, Molndal (SE); Staffan Karlsson, Molndal (SE); Peter Nordberg, Molndal (SE); Ingemar Starke, Molndal (SE)

Correspondence Address: Pepper Hamilton LLP, 400 Berwyn Park, 899 Cassatt Road, Berwyn, PA, 19312-1183, US

(73) Assignee: ASTRAZENECA AB, Sodertalje (SE)

(21) Appl. No.: 11/993,475

(22) PCT Filed: Jun. 21, 2006
(86) PCT No.: PCT/SE2006/000763

§ 371 Date: Dec. 21, 2007

(30) Foreign Application Priority Data
Jun. 22, 2005 (SE) 0501467-5

(51) Int. Cl. A61K 31/397; A61P 3/06; A61P 9/10; A61P 25/28; C07D 205/08

(52) U.S. Cl. 514/210.02; 540/360

(57) ABSTRACT

The invention relates to novel 2-azetidinone derivatives of formula (I) and to pharmaceutically acceptable salts, solvates and prodrugs thereof. The compounds are cholesterol absorption inhibitors, useful in the treatment of hyperlipidaemic conditions. The invention also relates to processes for their manufacture and to pharmaceutical compositions containing them.

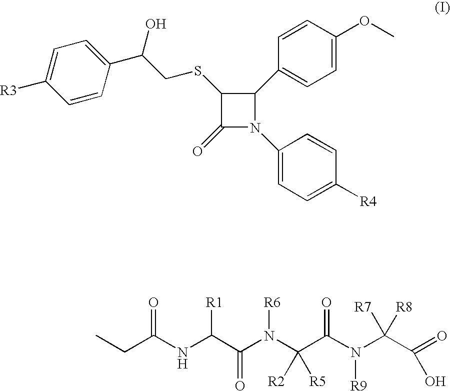

This invention relates to 2-azetidinone derivatives, or pharmaceutically acceptable salts, solvates, solvates of such salts and prodrugs thereof. These 2-azetidinones possess cholesterol absorption inhibitory activity and are accordingly of value in the treatment of disease states associated with hyperlipidaemic conditions. They are therefore useful in methods of treatment of a warm-blooded animal, such as man. The invention also relates to processes for the manufacture of said 2-azetidinone derivatives, to pharmaceutical compositions containing them and to their use in the manufacture of medicaments to inhibit cholesterol absorption in a warm-blooded animal, such as man. A further aspect of this invention relates to the use of the compounds of the invention in the treatment of dyslipidemic conditions.

Atherosclerotic coronary artery disease is a major cause of death and morbidity in the western world as well as a significant drain on healthcare resources. It is well-known that hyperlipidaemic conditions associated with elevated concentrations of total cholesterol and low density lipoprotein (LDL) cholesterol are major risk factors for cardiovascular atherosclerotic disease (for instance “Coronary. Heart Disease: Reducing the Risk; a Worldwide View” Assman G., Carmena R. Cullen P. et al; Circulation 1999, 100, 1930-1938 and “Diabetes and Cardiovascular Disease: A Statement for Healthcare Professionals from the American Heart Association” Grundy S, Benjamin I., Burke G., et al; Circulation, 1999, 100, 1134-46).

The concentration of plasma cholesterol depends on the integrated balance of endogenous and exogenous pathways of cholesterol metabolism. In the endogenous pathway, cholesterol is synthesized by the liver and extra hepatic tissues and enters the circulation as lipoproteins or is secreted into bile. In the exogenous pathway cholesterol from dietary and biliary sources is absorbed in the intestine and enters the circulation as component of chylomicrons. Alteration of either pathway will affect the plasma concentration of cholesterol.

The precise mechanism by which cholesterol is absorbed from the intestine is however not clear. The original hypothesis has been that cholesterol is crossing the intestine by unspecific diffusion. But more recent studies are suggesting that there are specific transporters involved in the intestinal cholesterol absorption. (See for instance New molecular targets for cholesterol-lowering therapy Izzat, N. N., Deshazer, M. E. and Loose-Mitchell D. S. JPET 293:315-320, 2000.)

A clear association between reduction of total cholesterol and (LDL) cholesterol and decreased instance of coronary artery disease has been established, and several classes of pharmaceutical agents are used to control serum cholesterol. There major options to regulate plasma cholesterol include (i) blocking the synthesis of cholesterol by agents such as HMG-CoA reductase inhibitors, for example statins such as simvastatin and fluvastatin, which also by up-regulation of LDL-receptors will promote the cholesterol removal from the plasma; (ii) blocking the bile acid reabsorption by specific agents resulting in increased bile acid excretion and synthesis of bile acids from cholesterol with agents such as bile acid binders, such as resins e.g. cholestyramine and cholestipol; and (iii) by blocking the intestinal uptake of cholesterol by selective cholesterol absorption inhibitors. High density lipoprotein (HDL) elevating agents such as fibrates and nicotinic acid analogues have also been employed.

Even with the current diverse range of therapeutic agents, a significant proportion of the hypercholesterolemic population is unable to reach target cholesterol levels, or drug interactions or drug safety preclude the long term use needed to reach the target levels. Therefore there is still a need to develop additional agents that are more efficacious and are better tolerated.

Compounds possessing such cholesterol absorption inhibitory activity have been described, see for instance the compounds described in WO 93/02048, WO 94/17038, WO 95/08532, WO 95/26334, WO 95/35277, WO 96/16037, WO 96/19450, WO 97/16455, WO 02/50027, WO 02/50060, WO 02/50068, WO 02/50090, WO 02/66464, WO 04/000803, WO 04/000804, WO04/000805, WO04/01993, WO04/010948, WO04/043456 WO 04/043457, WO 04/081002, WO05/000353, WO05/021495, WO05/021497, WO05/033100, U.S. Pat. No. 5,756,470, U.S. Pat. No. 5,767,115, US 20040180860, US20040180861 and U.S. RE37721.

The present invention is based on the discovery that certain 2-azetidinone derivatives surprisingly inhibit cholesterol absorption. Such properties are expected to be of value in the treatment of disease states associated with hyperlipidaemic conditions. The compounds of the present invention are not disclosed in any of the above applications and we have surprisingly found that the compounds of the present invention possess beneficial efficacious, metabolic and toxicological profiles that make them particularly suitable for in vivo administration to a warm blooded animal, such as man. In particular certain compounds of the present invention have a low degree of absorption compared to compounds of the prior art whilst retaining their ability to inhibit cholesterol absorption.

Accordingly there is provided a compound of formula (I):

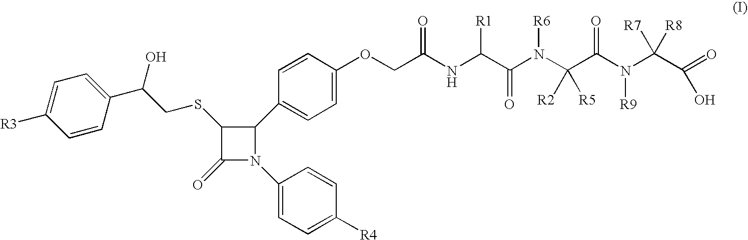

wherein:
R¹ is hydrogen, C₁₋₆alkyl, C₃₋₆cycloalkyl or aryl;
R², R⁵, R⁷ and R⁸ are independently hydrogen, a branched or unbranched C₁₋₆alkyl, C₃₋₆cycloalkyl or aryl; wherein said C₁₋₆alkyl may be optionally substituted by one or more hydroxy, amino, guanidino, cyano, carbamoyl, carboxy, C₁₋₆alkoxy, aryl C₁₋₆alkoxy, (C₁-C₄alkyl)₃Si, N—(C₁₋₆alkyl)amino, N,N—(C₁₋₆alkyl)₂-amino, C₁₋₆alkylS(O)ₐ, C₃₋₆cycloalkyl, aryl or aryl C₁₋₆ alkylS(O)ₐ, wherein a is 0-2; and wherein any aryl group may be optionally substituted by one or two substituents selected from halo, hydroxy, C₁₋₆alkyl, C₁₋₆alkoxy, or cyano;
R³ is hydrogen, alkyl, halo, C₁₋₆alkoxy or C₁₋₆ alkylS—;
R⁴ is hydrogen, C₁₋₆ alkyl, halo or C₁₋₆alkoxy;
R⁶ and R⁹ is hydrogen, C₁₋₆ alkyl, or arylC₁₋₆ alkyl;
wherein R⁵ and R² may form a ring with 2-7 carbon atoms and wherein R⁶ and R² may form a ring with 3-6 carbon atoms;
or a pharmaceutically acceptable salt, solvate, solvate of such a salt or a prodrug thereof.

In one aspect of the invention it is provided for a compound of formula I2:

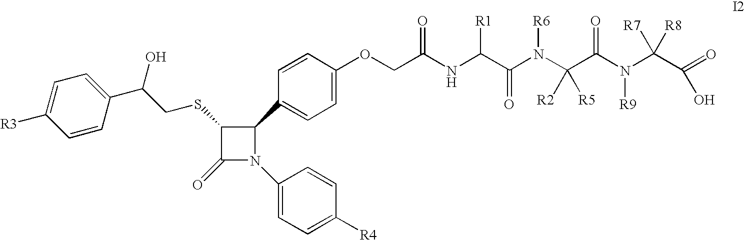

wherein variable groups are defined above as for formula (I). What is said further for formula (I) will, apart from the process schemes below, apply also to formula (I2).

According to one aspect of the invention R¹ is hydrogen;

R², R⁵, R⁷ and R⁸ are independently, hydrogen, a branched or unbranched C₁₋₆alkyl, C₃₋₆cycloalkyl or aryl; wherein said C₁₋₆alkyl may be optionally substituted by one or more hydroxy, amino, carbamoyl, carboxy, C₁₋₆alkoxy, aryl C₁₋₆alkoxy, (C₁-C₄alkyl)₃Si, N—(C₁₋₆alkyl)amino, N,N—(C₁₋₆alkyl)₂-amino, C₃₋₆cycloalkyl, aryl; and wherein any aryl group may be optionally substituted by one or two substituents selected from halo, hydroxy, C₁₋₆alkyl, C₁₋₆alkoxy, or cyano;
R³ is hydrogen, alkyl, halo or C₁₋₆alkoxy
R⁴ is hydrogen, C₁₋₆ alkyl, halo or C₁₋₆alkoxy;
R⁶ is hydrogen and R⁹ is hydrogen, C₁₋₆ alkyl, or arylC₁₋₆ alkyl.

According to an aspect of the invention, a compound according to the invention is chosen from one of the following compounds:
N-{(2R)-2-[(N-{[4-((2R,3R)-1-(4-fluorophenyl)-3-{[2-(4-fluorophenyl)-2-hydroxyethyl]thio}-4-oxoazetidin-2-yl)phenoxy]acetyl}glycyl)amino]-2-phenylacetyl}glycine;
N-{[4-((2R,3R)-1-(4-fluorophenyl)-3-{[2-(4-fluorophenyl)-2-hydroxyethyl]thio}-4-oxoazetidin-2-yl)phenoxy]acetyl}glycylglycyl-N-benzylglycine;
N-{[4-((2R,3R)-1-(4-fluorophenyl)-3-{[2-(4-fluorophenyl)-2-hydroxyethyl]thio}-4-oxoazetidin-2-yl)phenoxy]acetyl}glycylglycyl-N-ethylglycine;
N-{[4-((2R,3R)-1-(4-fluorophenyl)-3-{[2-(4-fluorophenyl)-2-hydroxyethyl]thio}-4-oxoazetidin-2-yl)phenoxy]acetyl}glycylglycyl-3-methyl-D-valine.hydrogen;
N-{[4-((2R,3R)-1-(4-fluorophenyl)-3-{[(2R or S)-2-(4-fluorophenyl)-2-hydroxyethyl]thio}-4-oxoazetidin-2-yl)phenoxy]acetyl}glycyl-3-methyl-D-valylglycine;
N-{[4-((2R,3R)-1-(4-fluorophenyl)-3-{[(2R)-2-(4-fluorophenyl)-2-hydroxyethyl]thio}-4-oxoazetidin-2-yl)phenoxy]acetyl}glycyl-3-methyl-D-valyl-D-serine;
N-{[4-((2R,3R)-1-(4-fluorophenyl)-3-{[(2R)-2-(4-fluorophenyl)-2-hydroxyethyl]thio}-4-oxoazetidin-2-yl)phenoxy]acetyl}glycyl-3-cyclohexyl-D-alanylglycine;
N-{[4-((2R,3R)-1-(4-fluorophenyl)-3-{[(2R)-2-(4-fluorophenyl)-2-hydroxyethyl]thio}-4-oxoazetidin-2-yl)phenoxy]acetyl}glycyl-3-cyclohexyl-D-alanylglycine;
N-{[4-((2R,3R)-1-(4-fluorophenyl)-3-{[(2R)-2-(4-fluorophenyl)-2-hydroxyethyl]thio}-4-oxoazetidin-2-yl)phenoxy]acetyl}glycyl-3-cyclohexyl-D-alanyl-D-alanine;
N-{[4-((2R,3R)-1-(4-fluorophenyl)-3-{[(2R)-2-(4-fluorophenyl)-2-hydroxyethyl]thio}-4-oxoazetidin-2-yl)phenoxy]acetyl}glycyl-N—[(R)-carboxy(phenyl)methyl]-3-cyclohexyl-D-alaninamide;
N-{[4-((2R,3R)-1-(4-fluorophenyl)-3-{[(2R)-2-(4-fluorophenyl)-2-hydroxyethyl]thio}-4-oxoazetidin-2-yl)phenoxy]acetyl}glycyl-3-cyclohexyl-D-alanyl-D-valine; and
N-{[4-((2R,3R)-1-(4-fluorophenyl)-3-{[(2R)-2-(4-fluorophenyl)-2-hydroxyethyl]thio}-4-oxoazetidin-2-yl)phenoxy]acetyl}glycyl-3-cyclohexyl-D-alanyl-D-lysine.

In this specification the term “alkyl” includes both straight and branched chain alkyl groups but references to individual alkyl groups such as “propyl” are specific for the straight chain version only. For example, “C₁₋₆alkyl” and “C₁₋₆alkyl” include propyl, isopropyl and t-butyl. However, references to individual alkyl groups such as ‘propyl’ are specific for the straight-chained version only and references to individual branched chain alkyl groups such as ‘isopropyl’ are specific for the branched chain version only. A similar convention applies to other radicals, for example “phenylC₁₋₆alkyl” would include benzyl, 1-phenylethyl and 2-phenylethyl. The term “halo” refers to fluoro, chloro, bromo and iodo.

Where optional substituents are chosen from “one or more” groups it is to be understood that this definition includes all substituents being chosen from one of the specified groups or the substituents being chosen from two or more of the specified groups.

The term “aryl” refers to a 4-10 membered aromatic mono or bicyclic ring containing 0 to 5 heteroatoms independently selected from nitrogen, oxygen or sulphur. Examples of aryls include phenyl, pyrrolyl, furanyl, imidazolyl, triazolyl, tetrazolyl, pyrazinyl, pyrimidinyl, pyridazinyl, pyridyl, isoxazolyl, oxazolyl, 1,2,4 oxadiazolyl, isothiazolyl, thiazolyl, 1,2,4-triazolyl, thienyl, naphthyl, benzofuranyl, benzimidazolyl, benzthienyl, benzthiazolyl, benzisothiazolyl, benzoxazolyl, benzisoxazolyl, 1,3-benzodioxolyl, indolyl, pyridoimidazolyl, pyrimidoimidazolyl, quinolyl, isoquinolyl, quinoxalinyl, quinazolinyl, phthalazinyl, cinnolinyl and naphthyridinyl. Particularly “aryl” refers to phenyl, thienyl, pyridyl, imidazolyl or indolyl. The term“aryl” includes both unsubstituted and substituted aromatic rings.

Examples of “C₁₋₆alkoxy” include methoxy, ethoxy and propoxy. Examples of “C₁₋₆alkylS(O)ₐ wherein a is 0 to 2” include methylthio, ethylthio, methylsulphinyl, ethylsulphinyl, mesyl and ethylsulphonyl. Examples of “N—(C₁₋₆alkyl)amino” include methylamino and ethylamino. Examples of “N,N—(C₁₋₆alkyl)₂-amino” include di-N-methylamino, di-(N-ethyl)amino and N-ethyl-N-methylamino. “C₃₋₆cycloalkyl” refers to cyclopropyl, cyclobutyl, cyclopentyl and cyclohexyl.

A suitable pharmaceutically acceptable salt of a compound of the invention, or other compounds disclosed herein, is, for example, an acid-addition salt of a compound of the invention which is sufficiently basic, for example, an acid-addition salt with, for example, an inorganic or organic acid, for example hydrochloric, hydrobromic, sulphuric, phosphoric, trifluoroacetic, citric, acetate or maleic acid. In addition a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

The compounds of the formula (I), or other compounds disclosed herein, may be administered in the form of a pro-drug which is broken down in the human or animal body to give a compound of the formula (I). Examples of pro-drugs include in vivo hydrolysable esters and in vivo hydrolysable amides of a compound of the formula (I).

An in vivo hydrolysable ester of a compound of the formula (I), or other compounds disclosed herein, containing carboxy or hydroxy group is, for example, a pharmaceutically acceptable ester which is hydrolysed in the human or animal body to produce the parent acid or alcohol. Suitable pharmaceutically acceptable esters for carboxy include C₁₋₆alkoxymethyl esters for example methoxymethyl, C₁₋₆alkanoyloxymethyl esters for example pivaloyloxymethyl, phthalidyl esters, C₃₋₈cycloalkoxycarbonyloxyC₁₋₆alkyl esters for example 1-cyclohexylcarbonyloxyethyl; 1,3-dioxolen-2-onylmethyl esters for example 5-methyl-1,3-dioxolen-2-onylmethyl; and C₁₋₆alkoxycarbonyloxyethyl esters for example 1-methoxycarbonyloxyethyl and may be formed at any carboxy group in the compounds of this invention.

An in vivo hydrolysable ester of a compound of the formula (I), or other compounds disclosed herein, containing a hydroxy group includes inorganic esters such as phosphate esters and α-acyloxyalkyl ethers and related compounds which as a result of the in vivo hydrolysis of the ester breakdown to give the parent hydroxy group. Examples of α-acyloxyalkyl ethers include acetoxymethoxy and 2,2-dimethylpropionyloxy-methoxy. A selection of in vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl. Examples of substituents on benzoyl include morpholino and piperazine linked from a ring nitrogen atom via a methylene group to the 3- or 4-position of the benzoyl ring.

A suitable value for an in viva hydrolysable amide of a compound of the formula (I), or other compounds disclosed herein, containing a carboxy group is, for example, a N—C₁₋₆alkyl or N,N-di-C₁₋₆alkyl amide such as N-methyl, N-ethyl, N-propyl, N,N-dimethyl, N-ethyl-N-methyl or N,N-diethyl amide.

Some compounds of the formula (I) may have chiral centres and/or geometric isomeric centres (E- and Z-isomers), and it is to be understood that the invention encompasses all such optical, diastereoisomers and geometric isomers that possess cholesterol absorption inhibitory activity.

The invention relates to any and all tautomeric forms of the compounds of the formula (I) that possess cholesterol absorption inhibitory activity.

It is also to be understood that certain compounds of the formula (I) can exist in solvated as well as unsolvated forms such as, for example, hydrated forms. It is to be understood that the invention encompasses all such solvated forms which possess cholesterol absorption inhibitory activity.

Preferred aspects of the invention are those which relate to the compound of formula (I) or a pharmaceutically acceptable salt thereof.

Another aspect of the present invention provides a process for preparing a compound of formula (I) or a pharmaceutically acceptable salt, solvate, solvate of such a salt or a prodrug thereof which process (wherein variable groups are, unless otherwise specified, as defined in formula (I)) comprises of:

### Process 1) Reacting a Compound of Formula (II):

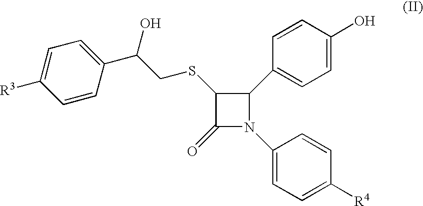

with a compound of formula (III):

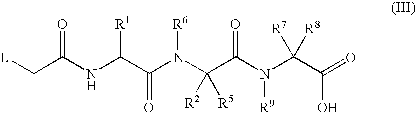

wherein L is a displaceable group;

### Process 2) Reacting an Acid of Formula (IV):

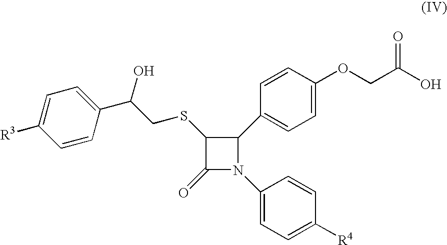

or an activated derivative thereof; with an amine of formula (V):

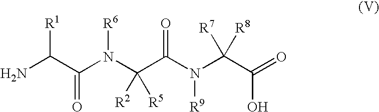

### Process 3): Reacting an Acid of Formula (VI):

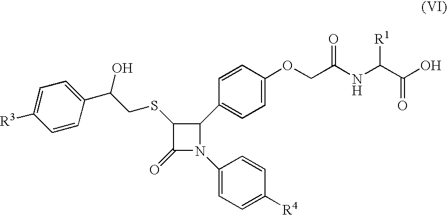

or an activated derivative thereof, with an amine of formula (VII):

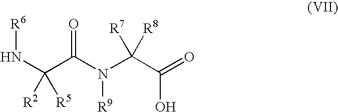

### Process 3a)): Reacting an Acid of Formula (VIb):

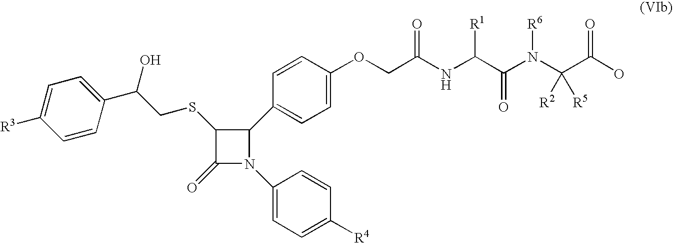

or an activated derivative thereof, with an amine of formula (VIIb):

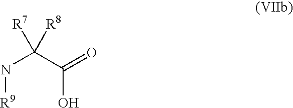

### Process 4): Reducing a Compound of Formula (VIII):

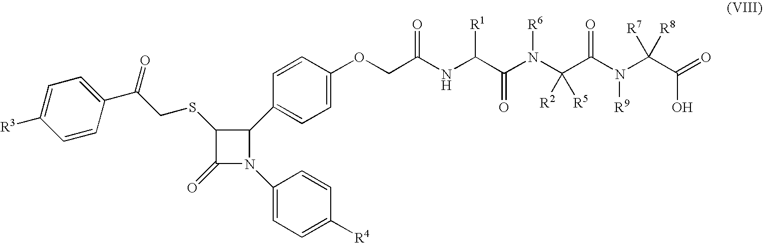

### Process 5): Reacting, a Compound of Formula (IX):

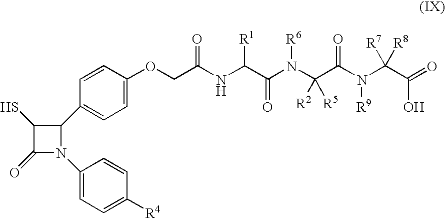

with a compound of formula (X):

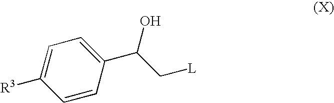

wherein L is a displaceable group;

### Process 6): Reacting a Compound of Formula (XI):

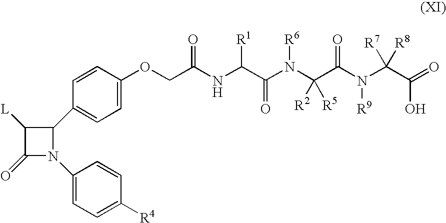

wherein L is a displaceable group; with a compound of formula (XII):

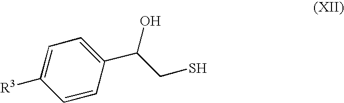

### Process 7): De-Esterifying a Compound of Formula (XIII)

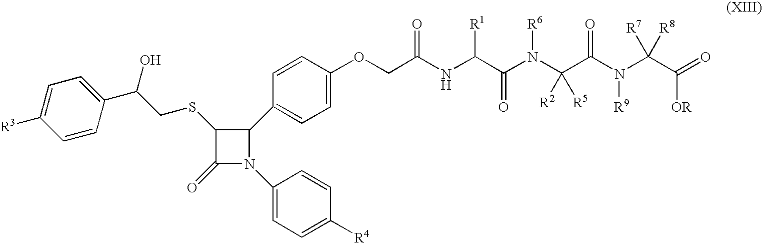

wherein the group C(O)OR is an ester group;
and thereafter if necessary or desirable:
i) converting a compound of the formula (I) into another compound of the formula (I);
ii) removing any protecting groups;
iii) forming a pharmaceutically acceptable salt, solvate, solvate of such a salt or a prodrug; or
iv) separating two or more enantiomers.

L is a displaceable group, suitable values for L are for example, a halogeno or sulphonyloxy group, for example a chloro, bromo, methanesulphonyloxy or toluene-4-sulphonyloxy group.

C(O)OR is an ester group, suitable values for C(O)OR are methoxycarbonyl, ethoxycarbonyl, t-butoxycarbonyl and benzyloxycarbonyl.

The starting materials used in the present invention can be prepared by modifications of the routes described in EP 0 792 264 B1. Alternatively they can be prepared by the following reactions.

Process 1): Alcohols of formula (II) may be reacted with compounds of formula (III) in the presence of a base for example an inorganic base such as sodium carbonate, or an organic base such as Hunigs base, in the presence of a suitable solvent such as acetonitrile, dichloromethane or tetrahydrofuran at a temperature in the range of 0° C. to reflux, preferably at or near reflux.

Compounds of formula (II) may be prepared according to the following scheme:

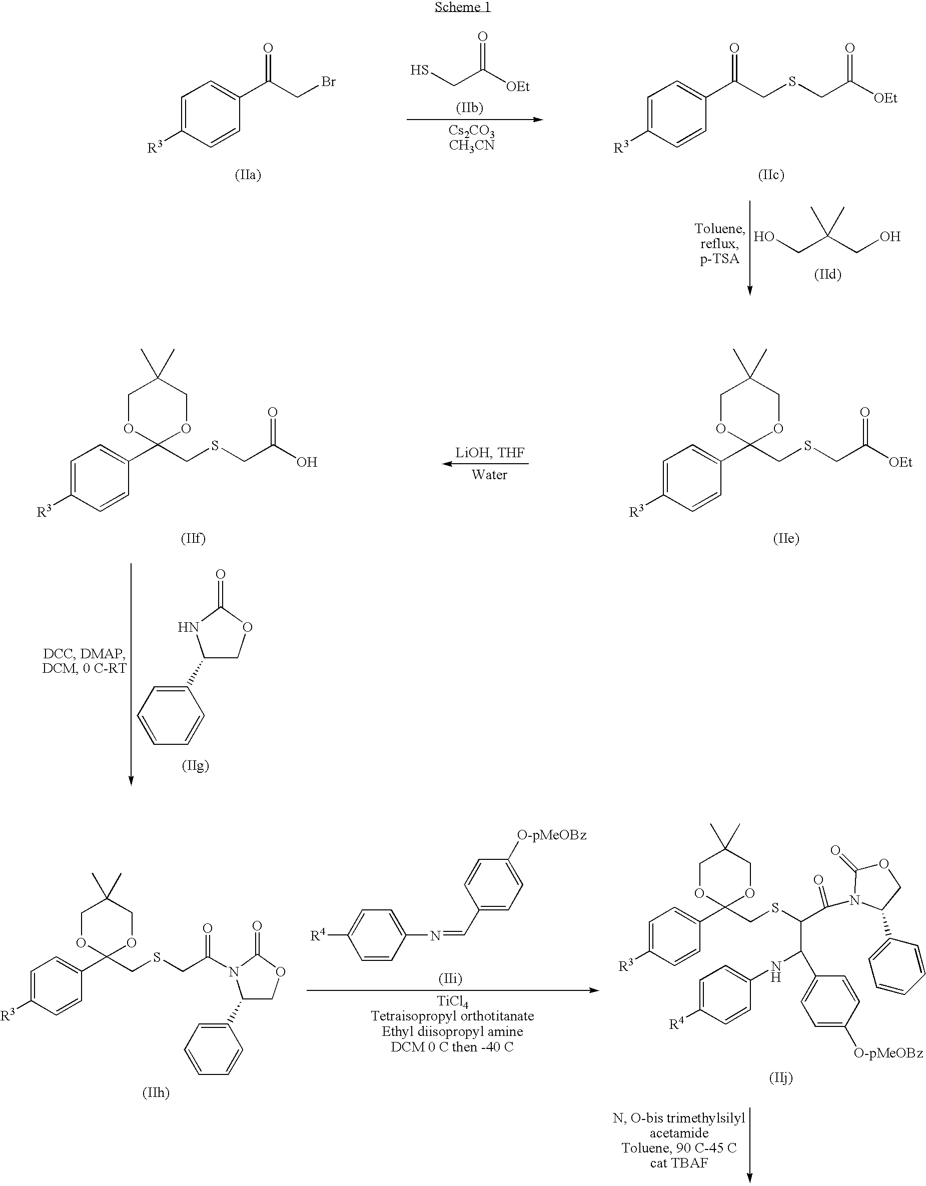

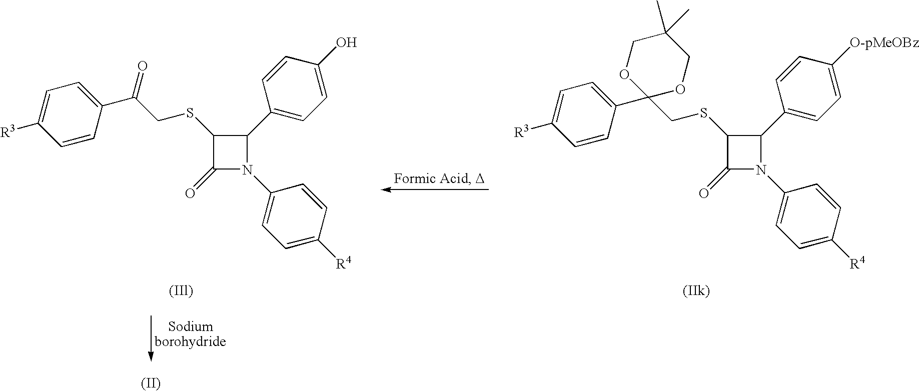

wherein pMeOBz is para methoxy benzyl.

Compounds of formula (IIb), (IId), (IIg) and (III) are commercially available compounds, or they are known in the literature, or they are prepared by standard processes known in the art.

Another aspect of the present invention provides a process for preparing a compound of formula (I2) or a pharmaceutically acceptable salt, solvate, solvate of such a salt or a prodrug thereof which process (wherein variable groups are, unless otherwise specified, as defined in formula (I)) comprises of:

### Process 1) Reacting a Compound of Formula (II2):

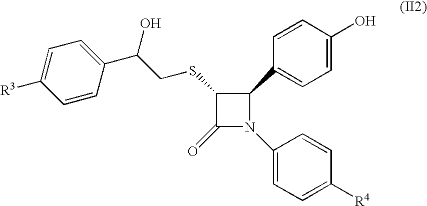

with a compound of formula (III):

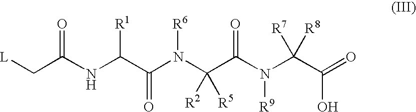

wherein L is a displaceable group;

### Process 2) Reacting an Acid of Formula (IV2):

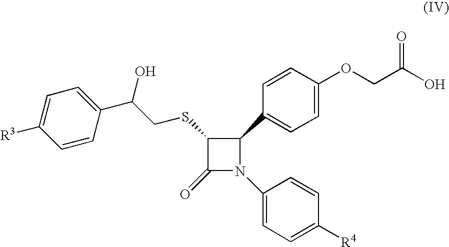

or an activated derivative thereof; with an amine of formula (V):

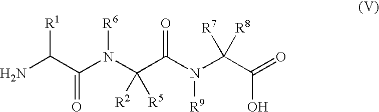

### Process 3): Reacting an Acid of Formula (VI2):

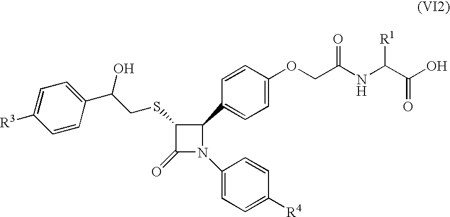

or an activated derivative thereof, with an amine of formula (VII):

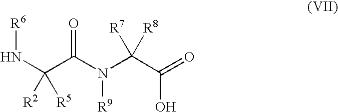

Process 3a)): reacting an acid of formula (VIb):

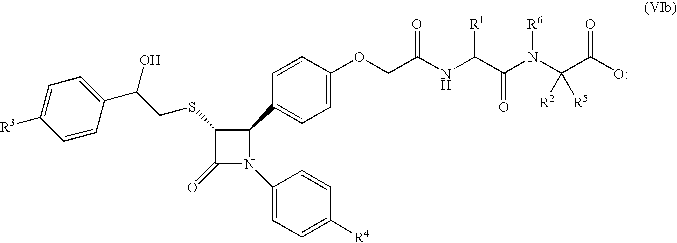

or an activated derivative thereof, with an amine of formula (VIIb):

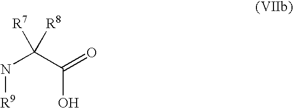

Process 4): reducing a compound of formula (VIII2):

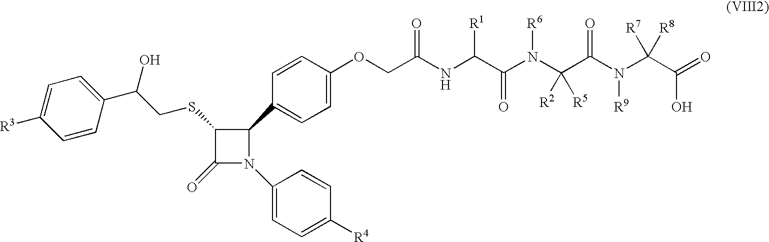

Process 5): reacting a compound of formula (IX2):

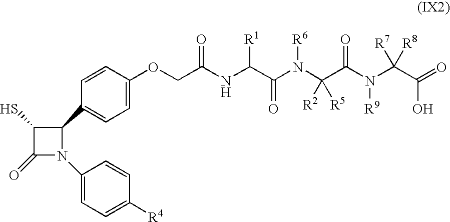

with a compound of formula (X):

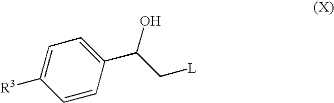

wherein L is a displaceable group;
Process 6): reacting a compound of formula (XI2):

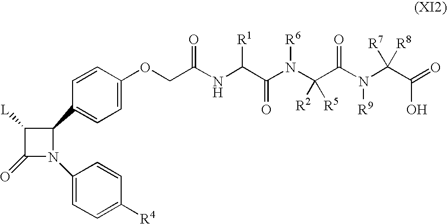

wherein L is a displaceable group; with a compound of formula (XII):

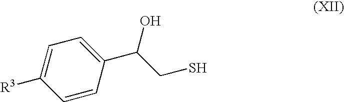

Process 7): De-esterifying a compound of formula (XIII2)

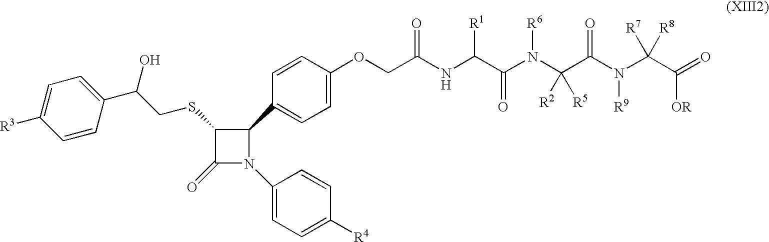

wherein the group C(O)OR is an ester group; and thereafter if necessary or desirable:
i) converting a compound of the formula (I2) into another compound of the formula (I2);
ii) removing any protecting groups;
iii) forming a pharmaceutically acceptable salt, solvate, solvate of such a salt or a prodrug; or
iv) separating two or more enantiomers.

L is a displaceable group, suitable values for L are for example, a halogeno or sulphonyloxy group, for example a chloro, bromo, methanesulphonyloxy or toluene-4-sulphonyloxy group.

C(O)OR is an ester group, suitable values for C(O)OR are methoxycarbonyl, ethoxycarbonyl, t-butoxycarbonyl and benzyloxycarbonyl.

The starting materials used in the present invention can be prepared by modifications of the routes described in EP 0 792 264 B1. Alternatively they can be prepared by the following reactions.

Process 1): Alcohols of formula (II2) may be reacted with compounds of formula (III) in the presence of a base for example an inorganic base such as sodium carbonate, or an organic base such as Hunigs base, in the presence of a suitable solvent such as acetonitrile, dichloromethane or tetrahydrofuran at a temperature in the range of 0° C. to reflux, preferably at or near reflux.

Compounds of formula (II2) may be prepared according to the following scheme:

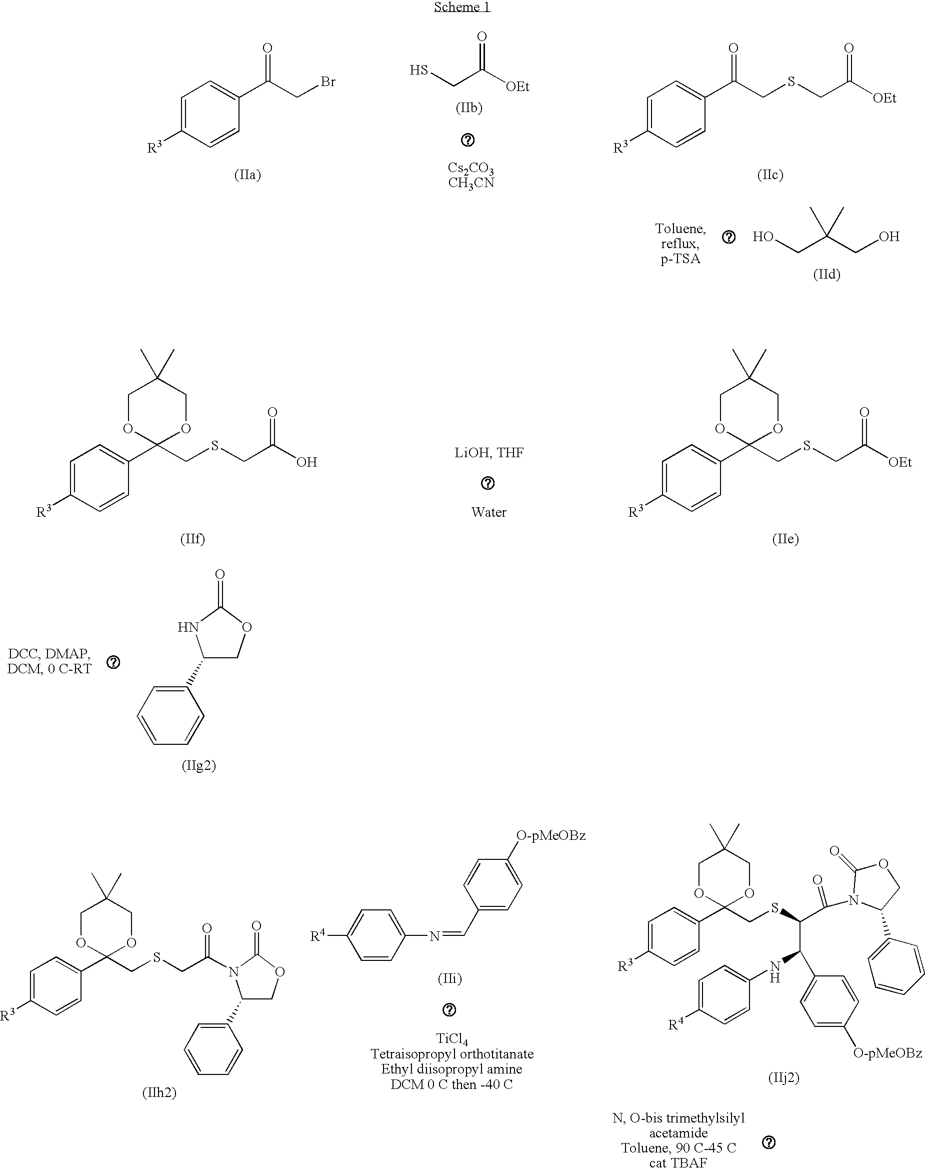

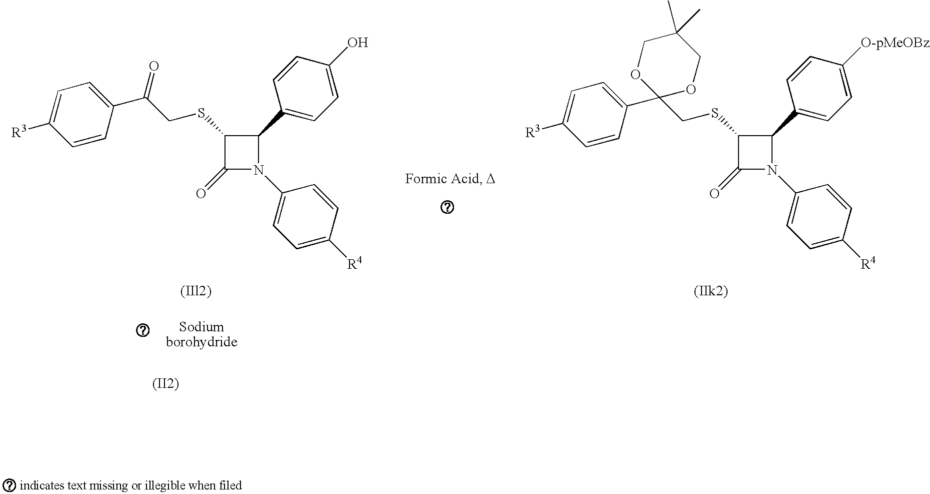

wherein pMeOBz is para methoxy benzyl.

Compounds of formula (IIb), (IId), (Iig2) and (III2) are commercially available compounds, or they are known in the literature, or they are prepared by standard processes known in the art.

A compound of formula (III) may also be reacted with a compound of formula (XIV).

Compounds of formula (XIV) may be prepared according to the following route:

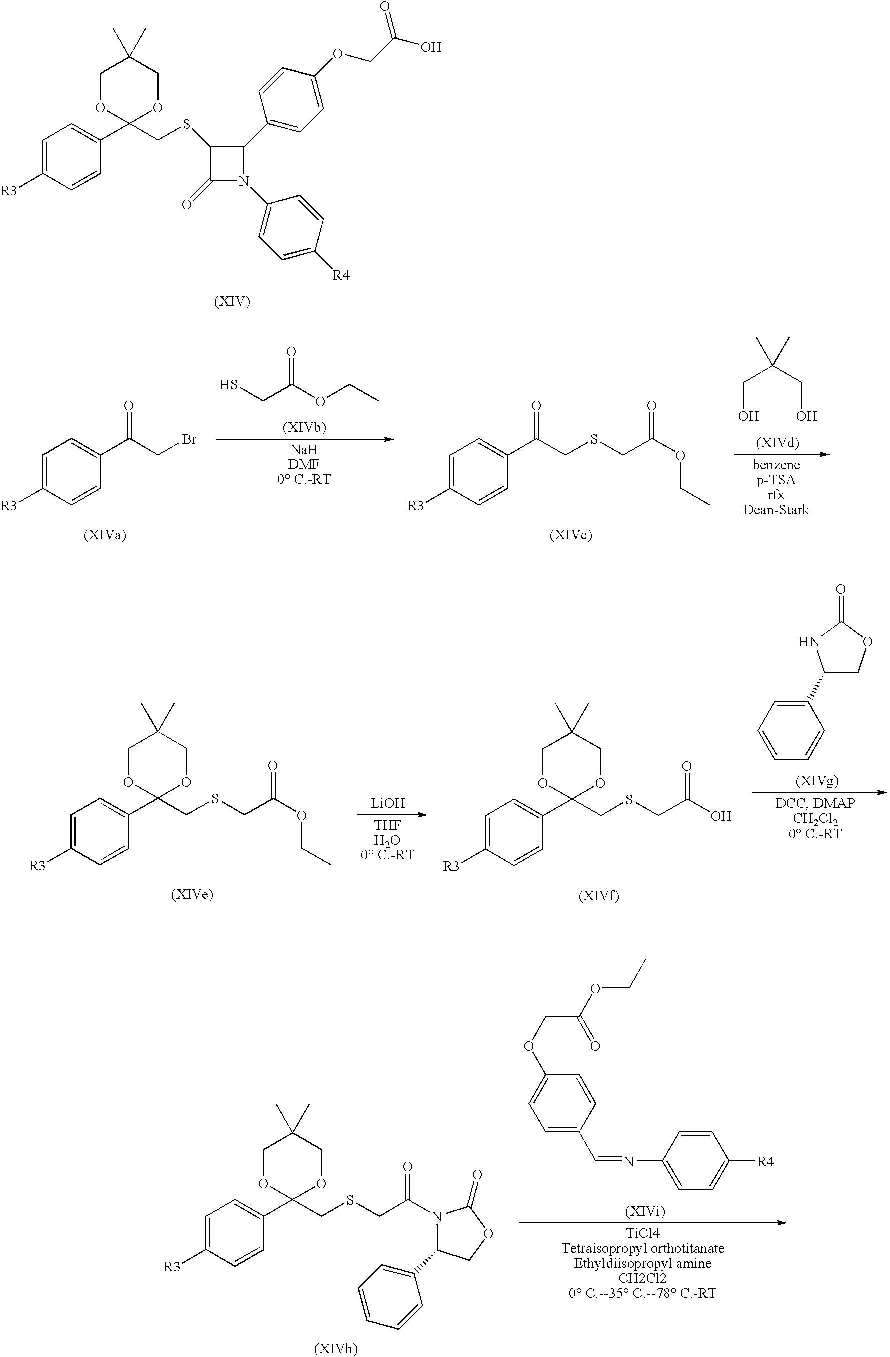

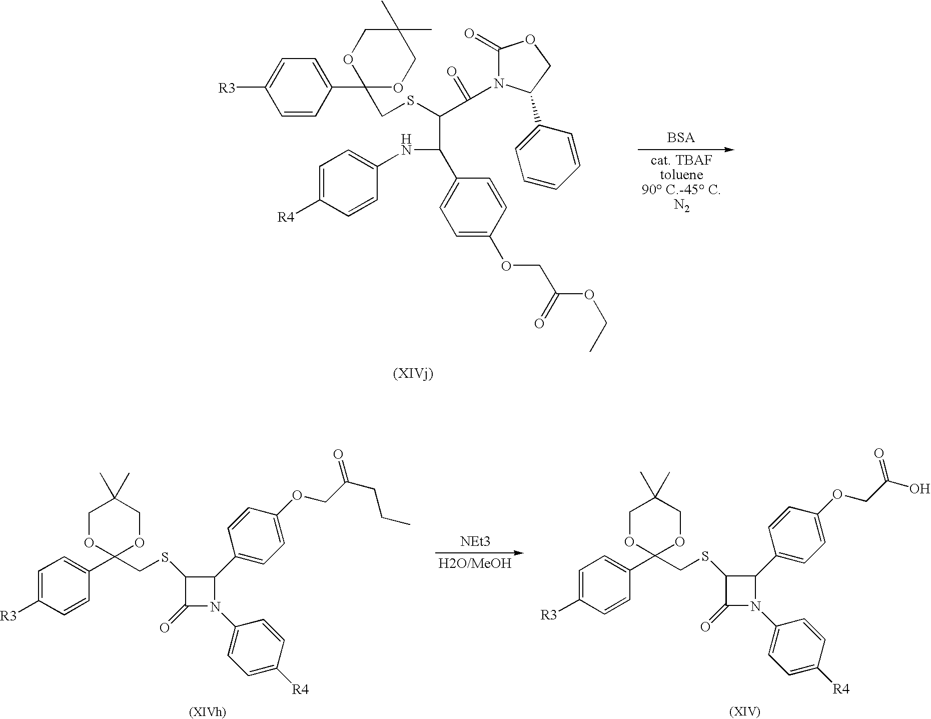

Compounds of formula XIVi may be prepared by the following route:

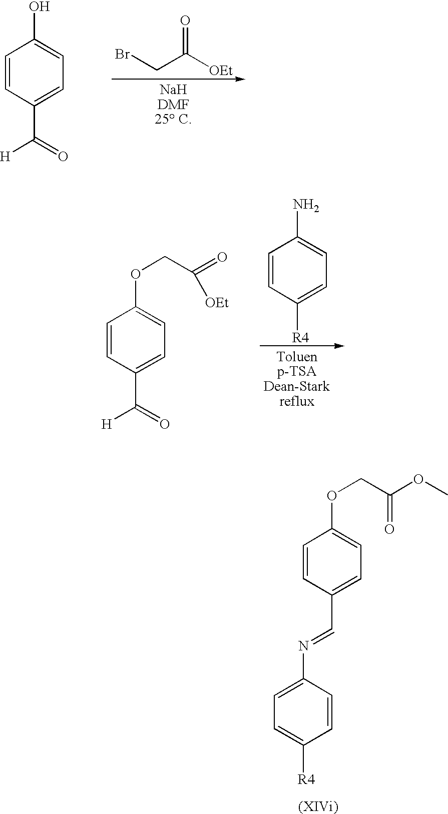

A compound of formula (III2) may also be reacted with a compound of formula (XIV2).

Compounds of formula (XIV2) may be prepared according to the following route:

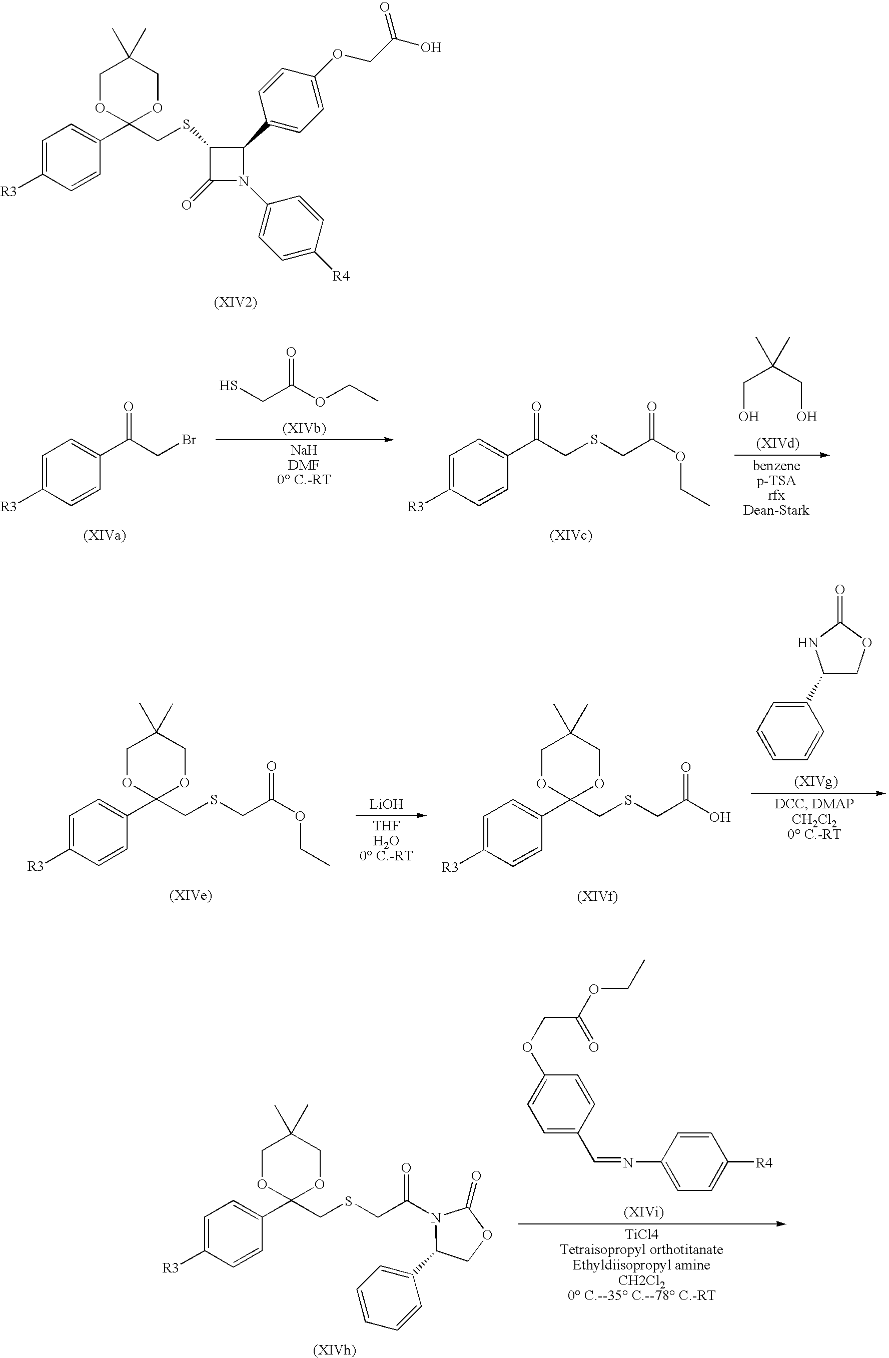

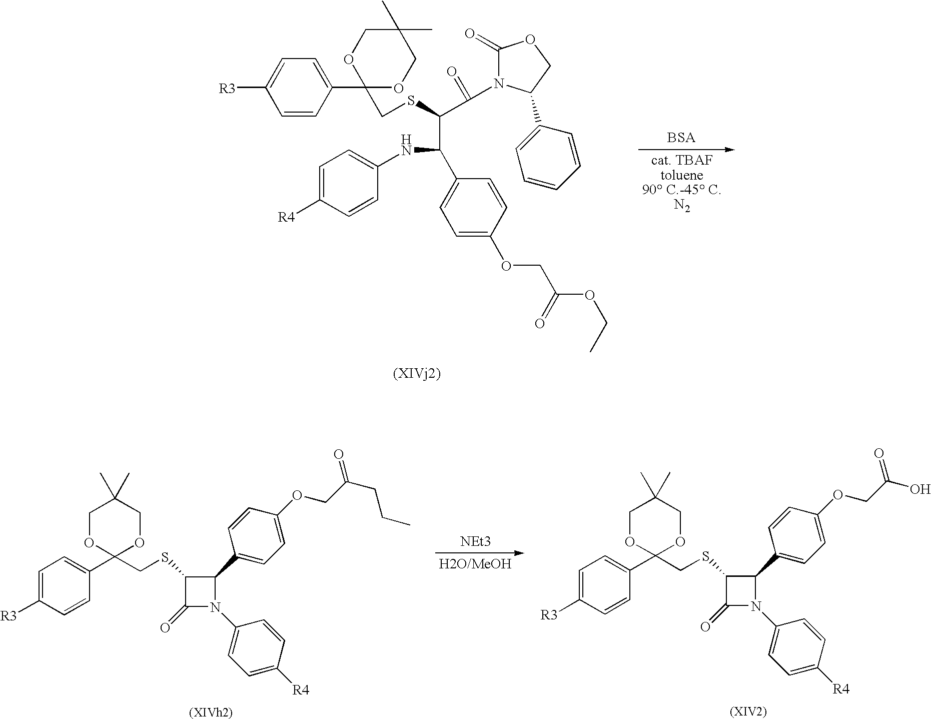

For XIV and XIV2 both, the following applies:

Process 2) and Process 3): Acids and amines may be coupled together in the presence of a suitable coupling reagent. Standard peptide coupling reagents known in the art can be employed as suitable coupling reagents, for example carbonyldiimidazole and dicyclohexyl-carbodiimide, optionally in the presence of a catalyst such as dimethylaminopyridine or 4-pyrrolidinopyridine, optionally in the presence of a base for example triethylamine, pyridine, or 2,6-di-alkyl-pyridines such as 2,6-lutidine or 2,6-di-tert-butylpyridine. Suitable solvents include dimethylacetamide, dichloromethane, benzene, tetrahydrofuran and dimethylformamide. The coupling reaction may conveniently be performed at a temperature in the range of −40 to 40° C.

Suitable activated acid derivatives include acid halides, for example acid chlorides, and active esters, for example pentafluorophenyl esters. The reaction of these types of compounds with amines is well known in the art, for example they may be reacted in the presence of a base, such as those described above, and in a suitable solvent, such as those described above. The reaction may conveniently be performed at a temperature in the range of −40 to 40° C.

Acids of formula (IV) and (VI) may be prepared from compounds of formula (II) by reacting them with the appropriate, optionally protected, side chain using the conditions of Process 1). Alternatively, acids of formula (IV) and (VI) may be prepared by a modification of Scheme I.

Amines of formula (V) and (VII) are commercially available compounds, or they are known in the literature, or they are prepared by standard processes known in the art.

Process 4): Reduction of compounds of formula (VIII) could be performed with a hydride reagent such as sodium borohydride in a solvent such as methanol at temperatures suitable between −20-40° C.

Compounds of formula (VIII) can be prepared from compounds of formula (III), by deprotecting the benzyl group and performing Process 1. Alternatively compound (IIk) could be debenzylated, Process 1 could be performed and the resulting compound deprotected to reveal the ketone.

Process 5) and Process 6): these compounds may be reacted together in the presence of a base for example an inorganic base such as sodium carbonate, or an organic base such as Hunigs base, in the presence of a suitable solvent such as acetonitrile, dichloromethane or tetrahydrofuran at a temperature in the range of 0° C. to reflux, preferably at or near reflux. Compounds of formula (IX) and (XI) may be prepared by an appropriate modification of Scheme 1.

Compounds of formula (X) and (XII) are commercially available compounds, or they are known in the literature, or they are prepared by standard processes known in the art.

Process 7): Esters of formula (XIII) may be deprotected under standard conditions such as those described below, for example a methyl or ethyl ester may be deprotected with sodium hydroxide in methanol at room temperature.

Compounds of formula (XIII) may be prepared by a modification of any of the processes described herein for the preparation of compounds of formula (I).

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminum trichloride) under Friedel Crafts conditions; the introduction of an alkyl group using an alkyl halide and Lewis acid (such as aluminum trichloride) under Friedel Crafts conditions; and the introduction of a halogeno group. Particular examples of modifications include the reduction of a nitro group to an amino group by for example, catalytic hydrogenation with a nickel catalyst or treatment with iron in the presence of hydrochloric acid with heating; oxidation of alkylthio to alkylsulphinyl or alkylsulphonyl.

It will also be appreciated that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Green, Protective Groups in Organic Synthesis, John Wiley and Sons, 1999). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

As stated hereinbefore the compounds defined in the present invention possess cholesterol absorption inhibitory activity. These properties may be assessed, using the following biological tests.

### In Vivo Testing of Cholesterol Absorption Inhibitors (A)

C57BL/6 female mice were maintained on regular chow diet and housed in individual cages to collect faeces. Mice were fasted for 3 hours and then gavaged with vehicle or compound. Half an hour later the mice were gavaged with radiolabelled cholesterol. Six hours after the ¹⁴C-cholesterol gavage blood samples were taken via the tail and plasma prepared to determine how much cholesterol were absorbed. 24 hours after the gavage of ¹⁴C-cholesterol the mice were bled and plasma were prepared for analysis. Faeces were collected for 24 hours to assess absorption efficiency.

### In Vivo Testing of Cholesterol Absorption Inhibitors (B).

C57BL/6 female mice were maintained on regular chow diet and housed in individual cages to collect faeces. Mice were fasted for 3 hours and then gavaged with vehicle or compound. One to ten hours later the mice were gavaged with radiolabelled cholesterol. Six hours after the ¹⁴C-cholesterol gavage blood sample was taken via the tail and plasma prepared to determine how much cholesterol was absorbed. 24 hours after the gavage of ¹⁴C-cholesterol the mice were bled and plasma analysed for radioactivity. Faeces were also collected for 24 hours to assess absorption efficiency.

### REFERENCES

1. E. A. Kirk, G. L. Moe, M. T. Caldwell, J. Å. Lemmark, D. L. Wilson, R. C. LeBoeuf. Hyper- and hypo-responsiveness to dietary fat and cholesterol among inbred mice: searching for level and variability genes. J. Lipid Res. 1995 36:1522-1532.
2. C. P. Carter, P. N. Howles, D. Y. Hui. Genetic variation in cholesterol absorption efficiency among inbred strains of mice. J. Nutr. 1997 127:1344-1348.
3. C. D. Jolley, J. M. Dietschy, S. D. Turley. Genetic differences in cholesterol absorption in 129/Sv and C57BL/6 mice: effect on cholesterol responsiveness. Am. J. Physiol. 1999 276:G1117-G1124.

Administration of 0.2 mmol/kg of Example 6 gave 49% inhibition of ¹⁴C-cholesterol absorption (procedure A). Administration of 0.2 μmol/kg of Example 7 gave 46% inhibition of ¹⁴C-cholesterol absorption (procedure A).

According to a further aspect of the invention there is provided a pharmaceutical composition which comprises a compound of formula (I), or a pharmaceutically acceptable salt, solvate, solvate of such a salt or a prodrug thereof, as defined hereinbefore in association with a pharmaceutically-acceptable diluent or carrier.

The composition may be in a form suitable for oral administration, for example as a tablet or capsule, for parenteral injection (including intravenous, subcutaneous, intramuscular, intravascular or infusion) as a sterile solution, suspension or emulsion, for topical administration as an ointment or cream or for rectal administration as a suppository.

In general the above compositions may be prepared in a conventional manner using conventional excipients.

The compound of formula (I), or a pharmaceutically acceptable salt, solvate, solvate of such a salt or a prodrug thereof, will normally be administered to a warm-blooded animal at a unit dose within the range of approximately 0.02-100 mg/kg, preferably 0.02-50 mg/kg, and this normally provides a therapeutically-effective dose. A unit dose form such as a tablet or capsule will usually contain, for example 1-250 mg of active ingredient. Preferably a daily dose in the range of 1-50 mg/kg, particularly 0.1-10 mg/kg is employed. In another aspect a daily dose in the rage of 0.01-20 mg/kg is employed. In one aspect of the invention the daily dose of a compound of formula (I) is less than or equal to 100 mg. However the daily dose will necessarily be varied depending upon the host treated, the particular route of administration, and the severity of the illness being treated. Accordingly the optimum dosage may be determined by the practitioner who is treating any particular patient.

According to a further aspect of the present invention there is provided a compound of the formula (I), or a pharmaceutically acceptable salt, solvate, solvate of such a salt or a prodrug thereof, as defined hereinbefore for use in a method of prophylactic or therapeutic treatment of a warm-blooded animal, such as man.

We have found that the compounds defined in the present invention, or a pharmaceutically acceptable salt, solvate, solvate of such a salt or a prodrug thereof, are effective cholesterol absorption inhibitors, and accordingly have value in the treatment of disease states associated with hyperlipidaemic conditions.

Thus according to this aspect of the invention there is provided a compound of the formula (I), or a pharmaceutically acceptable salt, solvate, solvate of such a salt or a prodrug thereof, as defined hereinbefore for use as a medicament.

According to another feature of the invention there is provided the use of a compound of the formula (I), or a pharmaceutically acceptable salt, solvate, solvate of such a salt or a prodrug thereof, as defined hereinbefore in the manufacture of a medicament for use in the production of a cholesterol absorption inhibitory effect in a warm-blooded animal, such as man.

According, to another feature of the invention there is provided the use of a compound of the formula (I), or a pharmaceutically acceptable salt, solvate, solvate of such a salt or a prodrug thereof, as defined hereinbefore in the production of a cholesterol absorption inhibitory effect in a warm-blooded animal, such as man.

Herein, where the production of a cholesterol absorption inhibitory effect or a cholesterol lowering effect is stated, suitably this relates to the treatment of hyperlipidaemic conditions in a warm-blooded animal, such as man. Additionally is relates to the treatment of dyslipidemic conditions and disorders such as hyperlipidaemia, hypertriglyceridemia, hyperbetalipoproteinemia (high LDL), hyperprebetalipoproteinemia (high VLDL), hyperchylomicronemia, hypolipoproteinemia, hypercholesterolemia, hyperlipoproteinemia and hypoalphalipoproteinemia (low HDL) in a warm-blooded animal, such as man. Furthermore it relates to the treatment of different clinical conditions such as atherosclerosis, arteriosclerosis, arrhythmia, hyper-thrombotic conditions, vascular dysfunction, endothelial dysfunction, heart failure, coronary heart diseases, cardiovascular diseases, myocardial infarction, angina pectoris, peripheral vascular diseases, inflammation of cardiovascular tissues such as heart, valves, vasculature, arteries and veins, aneurysms, stenosis, restenosis, vascular plaques, vascular fatty streaks, leukocytes, monocytes and/or macrophage infiltration, intimal thickening, medial thinning, infectious and surgical trauma and vascular thrombosis, stroke and transient ischaemic attacks in a warm-blooded animal, such as man. It also relates to the treatment of atherosclerosis, coronary heart diseases, myocardial infarction, angina pectoris, peripheral vascular diseases, stroke and transient ischaemic attacks in a warm-blooded animal, such as man.

The production of a cholesterol absorption inhibitory effect or a cholesterol lowering effect also relates to a method of treating and/or preventing atherosclerotic lesions, a method of preventing plaque rupture and a method of promoting lesion regression. Furthermore it relates to a method of inhibiting monocytes-macrophage accumulation in atherosclerotic lesions, a method of inhibiting expression of matrix metalloproteinases in atherosclerotic lesions, a method of inhibiting the destabilization of atherosclerotic lesions, a method for preventing atherosclerotic plaque rupture and a method of treating unstable angina.

The production of a cholesterol absorption inhibitory effect or a cholesterol lowering effect also relates to a method of treating sitosterolemia.

Compounds of formula (I), or a pharmaceutically acceptable salt, solvate, solvate of such a salt or a prodrug thereof may also have value in the treatment or prevention of Alzeheimer's Disease (see for example WO 02/096415). Therefore in a further aspect of the invention, there is provided a compound of formula (I), or a pharmaceutically acceptable salt, solvate, solvate of such a salt or a prodrug thereof, for use in the treatment or prevention of Alzheimer's Disease.

Compounds of formula (I), or a pharmaceutically acceptable salt, solvate, solvate of such a salt or a prodrug thereof may also have value in the treatment or prevention of cholesterol associated tumors. Therefore in a further aspect of the invention, there is provided a compound of formula (I), or a pharmaceutically acceptable salt, solvate, solvate of such a salt or a prodrug thereof, for use in the treatment or prevention of cholesterol associated tumors.

Compounds of formula (I), or a pharmaceutically acceptable salt, solvate, solvate of such a salt or a prodrug thereof may also have value in the treatment or prevention of vascular inflammation (see for example WO 03/026644). Therefore in a further aspect of the invention, there is provided a compound of formula (I), or a pharmaceutically acceptable salt, solvate, solvate of such a salt or a prodrug thereof, for use in the treatment or prevention of vascular inflammation.

According to a further feature of this aspect of the invention there is provided a method for producing a cholesterol absorption inhibitory effect in a warm-blooded animal, such as man, in need of such treatment which comprises administering to said animal an effective amount of a compound of formula (I), or a pharmaceutically acceptable salt, solvate, solvate of such a salt or a prodrug thereof.

The cholesterol absorption inhibitory activity defined hereinbefore may be applied as a sole therapy or may involve, in addition to a compound of the invention, one or more other substances and/or treatments. Such conjoint treatment may be achieved by way of the simultaneous, sequential or separate administration of the individual components of the treatment. According to this aspect of the invention there is provided a pharmaceutical product comprising a compound of the formula (I), or a pharmaceutically acceptable salt, solvate, solvate of such a salt or a prodrug thereof, as defined hereinbefore and an additional cholesterol absorption inhibitory substance as defined hereinbefore and an additional hypolipidaemic agent for the conjoint treatment of hyperlipidaemia.

In another aspect of the invention, the compound of formula (I), or a pharmaceutically acceptable salt, solvate, solvate of such a salt or a prodrug thereof, may be administered in association with cholesterol biosynthesis inhibitors, or pharmaceutically acceptable salts, solvates, solvates of such salts or prodrugs thereof. Suitable cholesterol biosynthesis inhibitors include HMG Co-A reductase inhibitors, squalene synthesis inhibitors and squalene epoxidase inhibitors. Suitable squalene synthesis inhibitors are e.g. squalestatin 1, TAK 475 and compounds described in WO2005012284. A suitable squalene epoxidase inhibitor is NB-598.

In this aspect of the invention, the compound of formula (I), or a pharmaceutically acceptable salt, solvate, solvate of such a salt or a prodrug thereof, may be administered in association with an HMG Co-A reductase inhibitor, or pharmaceutically acceptable salts, solvates, solvates of such salts or prodrugs thereof. Suitable HMG Co-A reductase inhibitors, pharmaceutically acceptable salts, solvates, solvates of such salts or prodrugs thereof are statins well known in the art. Particular statins are fluvastatin, lovastatin, pravastatin, simvastatin, atorvastatin, cerivastatin, bervastatin, dalvastatin, mevastatin and rosuvastatin, or a pharmaceutically acceptable salt, solvate, solvate of such a salt or a prodrug thereof. A further particular statin is pitavastatin, or a pharmaceutically acceptable salt, solvate, solvate of such a salt or a prodrug thereof. A particular statin is atorvastatin, or a pharmaceutically acceptable salt, solvate, solvate of such a salt or a prodrug thereof. A more particular statin is atorvastatin calcium salt. A further particular statin is rosuvastatin, or a pharmaceutically acceptable salt, solvate, solvate of such a salt or a prodrug thereof. A preferable particular statin is rosuvastatin calcium salt.

Therefore in an additional feature of the invention, there is provided a combination of a compound of formula (I), or a pharmaceutically acceptable salt, solvate, solvate of such a salt or a prodrug thereof and an HMG Co-A reductase inhibitor, or a pharmaceutically acceptable salt, solvate, solvate of such a salt or a prodrug thereof.

Therefore in an additional feature of the invention, there is provided a method for producing a cholesterol lowering effect in a warm-blooded animal, such as man, in need of such treatment which comprises administering to said animal an effective amount of a compound of formula (I), or a pharmaceutically acceptable salt, solvate, solvate of such a salt or a prodrug thereof in simultaneous, sequential or separate administration with an effective amount of an HMG Co-A reductase inhibitor, or a pharmaceutically acceptable salt, solvate, solvate of such a salt or a prodrug thereof.

According to a further aspect of the invention there is provided a pharmaceutical composition which comprises a compound of formula (I), or a pharmaceutically acceptable salt, solvate, solvate of such a salt or a prodrug thereof, and an HMG Co-A reductase inhibitor, or a pharmaceutically acceptable salt, solvate, solvate of such a salt or a prodrug thereof, in association with a pharmaceutically acceptable diluent or carrier.

According to a further aspect of the present invention there is provided a kit comprising a compound of formula (I), or a pharmaceutically acceptable salt, solvate, solvate of such a salt or a prodrug thereof, and an HMG Co-A reductase inhibitor, or a pharmaceutically acceptable salt, solvate, solvate of such a salt or a prodrug thereof.

According to a further aspect of the present invention there is provided a kit comprising:

a) a compound of formula (I), or a pharmaceutically acceptable salt, solvate, solvate of such a salt or a prodrug thereof, in a first unit dosage form;
b) an HMG Co-A reductase inhibitor, or a pharmaceutically acceptable salt, solvate, solvate of such a salt or a prodrug thereof; in a second unit dosage form; and
c) container means for containing said first and second dosage forms.

According to a further aspect of the present invention there is provided a kit comprising:

a) a compound of formula (I), or a pharmaceutically acceptable salt, solvate, solvate of such a salt or a prodrug thereof, together with a pharmaceutically acceptable diluent or carrier, in a first unit dosage form;
b) an HMG Co-A reductase inhibitor, or a pharmaceutically acceptable salt, solvate, solvate of such a salt or a prodrug thereof, in a second unit dosage form; and
c) container means for containing said first and second dosage forms.

According to another feature of the invention there is provided the use of a compound of the formula (I), or a pharmaceutically acceptable salt, solvate, solvate of such a salt or a prodrug thereof, and an HMG Co-A reductase inhibitor, or a pharmaceutically acceptable salt, solvate, solvate of such a salt or a prodrug thereof, in the manufacture of a medicament for use in the production of a cholesterol lowering effect.

According to a further aspect of the present, invention there is provided a combination treatment comprising the administration of an effective amount of a compound of the formula (I), or a pharmaceutically acceptable salt, solvate, solvate of such a salt or a prodrug thereof, optionally together with a pharmaceutically acceptable diluent or carrier, with the simultaneous, sequential or separate administration of an effective amount of an HMG Co-A reductase inhibitor, or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

According to an additional further aspect of the present invention there is provided a combination treatment comprising the administration of an effective amount of a compound of the formula (I), or a pharmaceutically acceptable salt, solvate, solvate of such a salt or a prodrug thereof, optionally together with a pharmaceutically acceptable diluent or carrier, with the simultaneous, sequential or separate administration of a matrix metalloproteinase inhibitor.

In another aspect of the invention, the compound of formula (I), or a pharmaceutically acceptable salt, solvate, solvate of such a salt or a prodrug thereof, may be administered in association with an ileal bile acid (MAT) inhibitor or a pharmaceutically acceptable salt, solvate, solvate of such a salt or a prodrug thereof. Suitable compounds possessing IBAT inhibitory activity for use in combination with compounds of the present invention have been described, see for instance the compounds described in WO 93/16055, WO 94/18183, WO 94/18184, WO 94/24087, WO 96/05188, WO 96/08484, WO 96/16051, WO 97/33882, WO 98/07749, WO 98138182, WO 98/40375, WO 98/56757, WO 99/32478, WO 99/35135, WO 99/64409, WO 99/64410, WO 00/01687, WO 00/20392, WO 00/20393, WO 00/20410, WO 00/20437, WO 00/35889, WO 01/34570, WO 00/38725, WO 00/38726, WO 00/38727, WO 00/38728, WO 00/38729, WO 00/47568, WO 00/61568, WO 01/66533, WO 01/68096, WO 01/68637, WO 02108211, WO 02/50051, WO 03/018024, WO 03/040127, WO 03/043992, WO 03/061604, WO 04/020421, WO 04/076430, DE 19825804, JP 10072371, U.S. Pat. No. 5,070,103, EP 251 315, EP 417 725, EP 489 423, EP 549 967, EP 573 848, EP 624 593, EP 624 594, EP 624 595, EP 864 582, EP 869 121 and EP 1 070 703, WO 03/020710, WO 03/022825, WO 03/022830, WO 03/022286, WO 03/091232, WO 03/106482, and EP 597 107

and the contents of these patent applications are incorporated herein by reference. Particularly the named examples of these patent applications are incorporated herein by reference. More particularly claim 1 of these patent application are incorporated herein by reference.

Other suitable classes of TEAT inhibitors for use in combination with compounds of the present invention are the benzothiepines, 1,2-benzothiazepines, 1,4-benzothiazepines and 1,5-benzothiazepines. A further suitable class of IBAT inhibitors is the 1,2,5-benzothiadiazepines.

One particular suitable compound possessing IBAT inhibitory activity for use in combination with compounds of the present invention is (3R,5R)-3-butyl-3-ethyl-1,1-dioxido-5-phenyl-2,3,4,5-tetrahydro-1,4-benzothiazepin-8-yl beta-D-glucopyranosiduronic acid (EP 864 582).

A further suitable compound possessing IBAT inhibitory activity for use in combination with compounds of the present invention is S-8921 (EP 597 107) and BARI-1741.

A further suitable IBAT inhibitor for use in combination with compounds of the present invention is the compound:

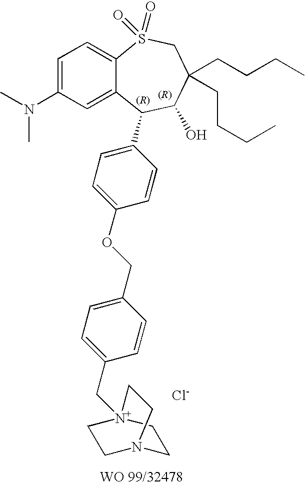

A particular IBAT inhibitor for use in combination with compounds of the present invention is selected from any one of Examples 1-120 of WO 02/50051, or a pharmaceutically acceptable salt, solvate, solvate of such a salt or a prodrug thereof, and the compounds of Examples 1-120 are incorporated herein by reference. Claims 1-15 of WO 02/50051 are also incorporated herein by reference. A particular IBAT inhibitor selected from WO 02/50051 for use in combination with compounds of the present invention is selected from any one of:
1,1-dioxo-3,3-dibutyl-5-phenyl-7-methylthio-8-(N—{(R)-1′-phenyl-1′-[N′-(carboxymethyl) carbamoyl]methyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′-(carboxymethyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N—{(R)-1′-phenyl-1′-[N′-(2-sulphoethyl)carbamoyl]methyl}carbamoylmethoxy)-2,3,4,5-tetrahydro-1,5-benzothiazepine;
1,1-dioxo-3-butyl-3-ethyl-5-phenyl-7-methylthio-8-(N—{(R)-1′-phenyl-1′-[N′-(2-sulphoethyl)carbamoyl]methyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′-(2-sulphoethyl)carbamoyl]-4-hydroxybenzyl}carbamoylmethoxy)-2,3,4,5-tetrahydro-1,5-benzothiazepine;
1,1-dioxo-3-butyl-3-ethyl-5-phenyl-7-methylthio-8-(N—{(R)-α-[N′-(2-sulphoethyl) carbamoyl]-4-hydroxybenzyl}carbamoylmethoxy)-2,3,4,5-tetrahydro-1,5-benzothiazepine;
1,1-dioxo-3-butyl-3-ethyl-5-phenyl-7-methylthio-8-(N—{(R)-α-[N′-(2-carboxyethypcarbamoyl]benzyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′-(2-carboxyethyl)carbamoyl]-4-hydroxybenzyl}carbamoylmethoxy)-2,3,4,5-tetrahydro-1,5-benzothiazepine;
1,1-dioxo-3-butyl-3-ethyl-5-phenyl-7-methylthio-8-(N—{(R)-α[N′-(5-carboxypentyl) carbamoyl]benzyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′-(2-carboxyethypcarbamoyl]benzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-(2-sulphoethyl)carbamoyl]-2-fluorobenzyl}carbamoylmethoxy)-2,3,4,5-tetrahydro-1,5-benzothiazepine;
1,1-dioxo-3-butyl-3-ethyl-5-phenyl-7-methylthio-8-(N—{(R)-α-[N′-(R)-(2-hydroxy-1-carboxyethyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′—(R)-(2-hydroxy-1-carboxyethyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-{N—[(R)-α-(N′-{(R)-1-[N″-(R)-(2-hydroxy-1-carboxyethyl)carbamoyl]-2-hydroxyethyl}carbamoyl)benzyl]carbamoylmethoxy}-2,3,4,5-tetrahydro-1,5-benzothiazepine;
1,1-dioxo-3-butyl-3-ethyl-5-phenyl-7-methylthio-8-(N-{α-[N′-(carboxymethyl)carbamoyl] benzyl}carbamoylmethoxy)-2,3,4,5-tetrahydro-1,5-benzothiazepine;
1,1-dioxo-3-butyl-3-ethyl-5-phenyl-7-methylthio-8-(N-{α-[N′-((ethoxy)(methyl)phosphoryl-methyl)carbamoyl]benzyl}carbamoylmethoxy)-2,3,4,5-tetrahydro-1,5-benzothiazepine;
1,1-dioxo-3-butyl-3-ethyl-5-phenyl-7-methylthio-8-{N—[(R)-α-(N′-{2-[(hydroxy)(methyl)phosphoryl]ethyl}carbamoyl)benzyl]carbamoylmethoxy}-2,3,4,5-tetrahydro-1,5-benzothiazepine;
1,1-dioxo-3,3-dibutyl-5-phenyl-7-methylthio-8-(N—{(R)-α-[N′-(2-methylthio-1-carboxyethyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-{N—[(R)-α-(N′-{2-[(methyl)(ethyl) phosphoryl]ethyl}carbamoyl)-4-hydroxybenzyl]carbamoylmethoxy}-2,3,4,5-tetrahydro-1,5-benzothiazepine;
1,1-dioxo-3,3-dibutyl-5-phenyl-7-methylthio-8-{N—[(R)-α-(N′-{2-[(methyl)(hydroxy) phosphoryl]ethyl}carbamoyl)-4-hydroxybenzyl]carbamoylmethoxy}-2,3,4,5-tetrahydro-1,5-benzothiazepine;
1,1-dioxo-3,3-dibutyl-5-phenyl-7-methylthio-8-(N—{(R)-α-[(R)—N′-(2-methylsulphinyl-1-carboxyethyl)carbamoyl]benzyl}carbamoylmethoxy)-2,3,4,5-tetrahydro-1,5-benzothiazepine; and
1,1-dioxo-3,3-dibutyl-5-phenyl-7-methoxy-8-[N—{(R)-α-[N-(2-sulphoethyl)carbamoyl]-4-hydroxybenzyl}carbamoylmethoxy]-2,3,4,5-tetrahydro-1,5-benzothiazepine;
   or a pharmaceutically acceptable salt, solvate, solvate of such a salt or a prodrug thereof.

A particular IBAT inhibitor for use in combination with compounds of the present invention is selected from any one of Examples 1-44 of WO 03/020710, or a pharmaceutically acceptable salt, solvate, solvate of such a salt or a prodrug thereof, and the compounds of Examples 1-44 are incorporated herein by reference. Claims 1-10 of WO 03/020710 are also incorporated herein by reference. A particular IBAT inhibitor selected from WO 03/020710 for use in combination with compounds of the present invention is selected from any one of:
1,1-dioxo-3,3-dibutyl-5-phenyl-7-methylthio-8-(N—{(R)-α-[N′-(2-(S)-3-(R)-4-(R)-5-(R)-2,3,4,5,6-pentahydroxyhexyl)carbamoyl]benzyl}carbamoylmethoxy)-2,3,4,5-tetrahydro-1,5-benzothiazepine;
1,1-dioxo-3-butyl-3-ethyl-5-phenyl-7-methylthio-8-(N—{(R)-α-[N′-(2-(S)-3-(R)-4-(R)-5-(R)-2,3,4,5,6-pentahydroxyhexyl)carbamoyl]benzyl}carbamoylmethoxy)-2,3,4,5-tetrahydro-1,5-benzothiazepine;
1,1-dioxo-3-butyl-3-ethyl-5-phenyl-7-methylthio-8-(N—{(R)-α-[N′-((S)-1-carbamoyl-2-hydroxyethyl)carbamoyl]benzyl}carbamoylmethoxy)-2,3,4,5-tetrahydro-1,5-benzothiazepine;
1,1-dioxo-3-butyl-3-ethyl-5-phenyl-7-methylthio-8-(N—{(R)-α-[N′-(hydroxycarbamoyl-methyl)carbamoyl]benzyl}carbamoylmethoxy)-2,3,4,5-tetrahydro-1,5-benzothiazepine;
1,1-dioxo-3-butyl-3-ethyl-5-phenyl-7-methylthio-8-[N—((R)-α-{N′-[2-(N′-pyrimidin-2-ylureido)ethyl]carbamoyl}benzyl)carbamoylmethoxy]-2,3,4,5-tetrahydro-1,5-benzothiazepine;
1,1-dioxo-3-butyl-3-ethyl-5-phenyl-7-methylthio-8-[N—((R)-α-{N′-[2-(N-pyridin-2-ylureido)ethyl]carbamoyl}benzyl)carbamoylmethoxy]-2,3,4,5-tetrahydro-1,5-benzothiazepine;
1,1-dioxo-3-butyl-3-ethyl-5-phenyl-7-methylthio-8-(N—{(R)-α-[N′-(1-t-butoxycarbonylpiperidin-4-ylmethyl)carbamoyl]benzyl}carbamoylmethoxy)-2,3,4,5-tetrahydro-1,5-benzothiazepine;
1,1-dioxo-3-butyl-3-ethyl-5-phenyl-7-methylthio-8-(N—{(R)-α-[N′-(2,3-dihydroxypropyl)carbamoyl]benzyl}carbamoylmethoxy)-2,3,4,5-tetrahydro-1,5-benzothiazepine;
1,1-dioxo-3-butyl-3-ethyl-5-phenyl-7-methylthio-8-[N—((R)-α-{N′-[2-(3,4-dihydroxyphenyl)-2-methoxyethyl]carbamoyl}benzyl)carbamoylmethoxy]-2,3,4,5-tetrahydro-1,5-benzothiazepine
1,1-dioxo-3-butyl-3-ethyl-5-phenyl-7-methylthio-8-(N—{(R)-α-[N′-(2-aminoethyl)carbamoyl]benzyl}carbamoylmethoxy)-2,3,4,5-tetrahydro-1,5-benzothiazepine;
1,1-dioxo-3-butyl-3-ethyl-5-phenyl-7-methylthio-8-(N—{(R)-α-[N′-(piperidin-4-ylmethyl) carbamoyl]benzyl}carbamoylmethoxy)-2,3,4,5-tetrahydro-1,5-benzothiazepine; or
1,1-dioxo-3-butyl-3-ethyl-5-phenyl-7-methylthio-8-(N—{(R)-α-[N′-(2-N,N-dimethylaminosulphamoylethyl)carbamoyl]benzyl}carbamoylmethoxy)-2,3,4,5-tetrahydro-1,5-benzothiazepine;
   or a pharmaceutically acceptable salt, solvate, solvate of such a salt or a prodrug thereof.

A particular IBAT inhibitor for use in combination with compounds of the present invention is selected from any one of Examples 1-7 of WO 03/022825, or a pharmaceutically acceptable salt, solvate, solvate of such a salt or a prodrug thereof, and the compounds of Examples 1-7 are incorporated herein by reference. Claims 1-8 of WO 03/022825 are also incorporated herein by reference. A particular IBAT inhibitor selected from WO 03/022825 for use in combination with compounds of the present invention is selected from any one of:
1,1-dioxo-3(R)-3-butyl-3-ethyl-5-(R)-5-phenyl-8-[N—((R)-α-carboxybenzyl) carbamoylmethoxy]-2,3,4,5-tetrahydro-1,4-benzothiazepine;
1,1-dioxo-3(S)-3-butyl-3-ethyl-5-(S)-5-phenyl-8-[N—((R)-α-carboxybenzyl) carbamoylmethoxy]-2,3,4,5-tetrahydro-1,4-benzothiazepine;
1,1-dioxo-3(R)-3-butyl-3-ethyl-5-(R)-5-phenyl-8-(N—{(R)-α-[N-(carboxymethyl)carbamoyl]benzyl}carbamoylmethoxy)-2,3,4,5-tetrahydro-1,4-benzothiazepine;
1,1-dioxo-3(S)-3-butyl-3-ethyl-5-(S)-5-phenyl-8-(N—{(R)-α-[N-(carboxymethyl)carbamoyl]benzyl}carbamoylmethoxy)-2,3,4,5-tetrahydro-1,4-benzothiazepine;
3,5-trans-1,1-dioxo-3-ethyl-3-butyl-5-phenyl-7-bromo-8-(N—{(R)-α-[N-(carboxymethyl)carbamoyl]benzyl}carbamoylmethoxy)-2,3,4,5-tetrahydro-1,4-benzothiazepine;
3,5-trans-1,1-dioxo-3-(S)-3-ethyl-3-butyl-4-hydroxy-5-(S)-5-phenyl-7-bromo-8-(N—{(R)-α-[N-(carboxymethyl)carbamoyl]benzyl}carbamoylmethoxy)-2,3,4,5-tetrahydro-1,4-benzothiazepine
3,5-trans-1,1-dioxo-3-(R)-3-ethyl-3-butyl-4-hydroxy-5-(R)-5-phenyl-7-bromo-8-(N—{(R)-α-[N-(carboxymethyl)carbamoyl]benzyl}carbamoylmethoxy)-2,3,4,5-tetrahydro-1,4-benzothiazepine;
3,5-trans-1,1-dioxo-3-ethyl-3-butyl-5-phenyl-7-methylthio-8-(N—{(R)-α-[N-(carboxymethyl)carbamoyl]benzyl}carbamoylmethoxy)-2,3,4,5-tetrahydro-1,4-benzothiazepine;
3,5-trans-1,1-dioxo-3-ethyl-3-butyl-5-phenyl-7-methylthio-8-(N—{(R)-α-[N-(2-sulphoethyl)carbamoyl]-4-hydroxybenzyl}carbamoylmethoxy)-2,3,4,5-tetrahydro-1,4-benzothiazepine ammonia salt;
1,1-dioxo-3-(S)-3-ethyl-3-butyl-5-(S)-5-phenyl-7-methylthio-8-(N—{(R)-α-[N-(carboxymethyl)carbamoyl]benzyl}carbamoylmethoxy)-2,3,4,5-tetrahydro-1,4-benzothiazepine diethylamine salt; and
1,1-dioxo-3-(R)-3-ethyl-3-butyl-5-(R)-5-phenyl-7-methylthio-8-(N—{(R)-α-[N-(carboxymethyl)carbamoyl]benzyl}carbamoylmethoxy)-2,3,4,5-tetrahydro-1,4-benzothiazepine diethylamine salt;
   or a pharmaceutically acceptable salt, solvate, solvate of such a salt or a prodrug thereof.

A particular IBAT inhibitor for use in combination with compounds of the present invention is selected from any one of Examples 1-4 of WO 03/022830, or a pharmaceutically acceptable salt, solvate, solvate of such a salt or a prodrug thereof, and the compounds of Examples 1-4 are incorporated herein by reference. Claims 1-8 of WO 03/022830 are also incorporated herein by reference. A particular IBAT inhibitor selected from WO 03/022830 for use in combination with compounds of the present invention is selected from any one of:
1,1-dioxo-3-butyl-3-ethyl-4-hydroxy-5-phenyl-7-(N—{(R)-α-[N-(carboxymethyl)carbamoyl]benzyl}carbamoylmethylthio)-2,3,4,5-tetrahydrobenzothiepine
1,1-dioxo-3-butyl-3-ethyl-4-hydroxy-5-phenyl-7-(N—{(R)-α-[N-(2-sulphoethyl)carbamoyl]-4-hydroxybenzyl}carbamoylmethylthio)-2,3,4,5-tetrahydrobenzothiepine ammonia salt
1,1-dioxo-3-butyl-3-ethyl-4-hydroxy-5-phenyl-7-{N-[α-(carboxy)-2-fluorobenzyl]carbamoylmethylthio}-2,3,4,5-tetrahydrobenzothiepine; and
1,1-dioxo-3-butyl-3-ethyl-4-hydroxy-5-phenyl-7-{N-[1-(carboxy)-1-(thien-2-yl)methyl]carbamoylmethylthio}-2,3,4,5-tetrahydrobenzothiepine
   or a pharmaceutically acceptable salt, solvate, solvate of such a salt or a prodrug thereof.

A particular IBAT inhibitor for use in combination with compounds of the present invention is selected from any one of Examples 1-39 of WO 03/022286, or a pharmaceutically acceptable salt, solvate, solvate of such a salt or a prodrug thereof, and the compounds of Examples 1-39 are incorporated herein by reference. Claims 1-10 of WO 03/022286 are also incorporated herein by reference. A particular IBAT inhibitor selected from WO 03/022286 for use in combination with compounds of the present invention is selected from any one of:
1,1-dioxo-3,3-dibutyl-5-phenyl-7-methylthio-8-(N—{(R)-α-[N—((R)-1-carboxy-2-methylthioethyl)carbamoyl]-4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N—{(R)-α-[N—((S)-1-carboxy-2-(R)-hydroxypropyl)carbamoyl]-4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N—{(R)-α-[N—((S)-1-carboxy-2-methylpropyl)carbamoyl]-4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N—{(R)-α-[N—((S)-1-carboxybutyl) carbamoyl]-4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N—{(R)-α-[N—((S)-1-carboxypropyl) carbamoyl]benzyl}carbamoylmethoxy)-2,3,4,5-tetrahydro-1,2,5-benzothiadiazepine;
1,1-dioxo-3,3-dibutyl-5-phenyl-7-methylthio-8-(N—{(R)-α-[N—((S)-1-carboxyethyl) carbamoyl]benzyl}carbamoylmethoxy)-2,3,4,5-tetrahydro-1,2,5-benzothiadiazepine;
1,1-dioxo-3,3-dibutyl-5-phenyl-7-methylthio-8-(N—{(R)-α-[N—((S)-1-carboxy-2-(R)-hydroxypropyl)carbamoyl]benzyl}carbamoylmethoxy)-2,3,4,5-tetrahydro-1,2,5-benzothiadiazepine;
1,1-dioxo-3,3-dibutyl-5-phenyl-7-methylthio-8-(N—{(R)-α-[N-(2-sulphoethyl)carbamoyl]-4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N—{(R)-α-[N—((S)-1-carboxyethyl)carbamoyl]-4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N—{(R)-α-[N—((R)-1-carboxy-2-methylthioethyl)carbamoyl]benzyl}carbamoylmethoxy)-2,3,4,5-tetrahydro-1,2,5-benzothiadiazepine;
1,1-dioxo-3,3-dibutyl-5-phenyl-7-methylthio-8-(N—{(R)-α-[N-{(S)-1-[N—((S)-2-hydroxy-1-carboxyethyl)carbamoyl]propyl}carbamoyl]benzyl}carbamoylmethoxy)-2,3,4,5-tetrahydro-1,2,5-benzothiadiazepine;
1,1-dioxo-3,3-dibutyl-5-phenyl-7-methylthio-8-(N—{(R)-α-[N—((S)-1-carboxy-2-methylpropyl)carbamoyl]benzyl}carbamoylmethoxy)-2,3,4,5-tetrahydro-1,2,5-benzothiadiazepine;
1,1-dioxo-3,3-dibutyl-5-phenyl-7-methylthio-8-(N—{(R)-α-[N—((S)-1-carboxypropyl) carbamoyl]-4-hydroxybenzyl}carbamoylmethoxy)-2,3,4,5-tetrahydro-1,2,5-benzothiadiazepine; and
1,1-dioxo-3,3-dibutyl-5-phenyl-7-methylthio-8-[N—((R)-α-carboxy-4-hydroxybenzyl)carbamoylmethoxy]-2,3,4,5-tetrahydro-1,2,5-benzothiadiazepine;
   or a pharmaceutically acceptable salt, solvate, solvate of such a salt or a prodrug thereof.

A particular IBAT inhibitor for use in combination with compounds of the present invention is selected from any one of Examples 1-7 of WO 03/091232, or a pharmaceutically acceptable salt, solvate, solvate of such a salt or a prodrug thereof, and the compounds of Examples 1-7 are incorporated herein by reference. Claims 1-10 of WO 03/091232 are also incorporated herein by reference. A particular IBAT inhibitor selected from WO 03/091232 for use in combination with compounds of the present invention is selected from any one of:
1,1-Dioxo-3,3-dibutyl-5-phenyl-7-methylthio-8-(N—{(R)-α-[N-(2-(S)-3-(R)-4-(R)-5-(R)-2,3,4,5,6-pentahydroxyhexyl)carbamoyl]benzyl}carbamoylmethoxy)-2,3,4,5-tetrahydro-1,2,5-benzothiadiazepine;
1,1-Dioxo-3,3-dibutyl-5-phenyl-7-methylthio-8-(N—{(R)-α-[N-(2-(S)-3-(R)-4-(R)-5-(R)-2,3,4,5,6-pentahydroxyhexyl)carbamoyl]-4-hydroxybenzyl}carbamoylmethoxy)-2,3,4,5-tetrahydro-1,2,5-benzothiadiazepine;
1,1-Dioxo-3,3-dibutyl-5-phenyl-7-methylthio-8-[N—((R/S)-α-{N-[1-(R)-2-(S)-1-hydroxy-1-(3,4-dihydroxyphenyl)prop-2-yl]carbamoyl}-4-hydroxybenzyl)carbamoylmethoxy]-2,3,4,5-tetrahydro-1,2,5-benzothiadiazepine;
1,1-Dioxo-3,3-dibutyl-5-phenyl-7-methylthio-8-{N—[(R)-α-(N-{2-(S)—[N-(carbamoylmethyl) carbamoyl]pyrrolidin-1-ylcarbonylmethyl}carbamoyl)benzyl]carbamoylmethoxy}-2,3,4,5-tetrahydro-1,2,5-benzothiadiazepine;
1,1-Dioxo-3,3-dibutyl-5-phenyl-7-methylthio-8-[N—((R)-α-{N′-[2-(3,4,5-trihydroxyphenyl)ethyl]carbamoyl}benzyl)carbamoylmethoxy]-2,3,4,5-tetrahydro-1,2,5-benzothiadiazepine; and
1,1-Dioxo-3,3-dibutyl-5-phenyl-7-methylthio-8-(N—{(R)-α[N-(2-(R)-3-(S)-4-(S)-5-(R)-3,4,5,6-tetrahydroxytetrahydropyran-2-ylmethyl)carbamoyl]benzyl}carbamoylmethoxy)-2,3,4,5-tetrahydro-1,2,5-benzothiadiazepine;
   or a pharmaceutically acceptable salt, solvate, solvate of such a salt or a prodrug thereof.

Further suitable compounds possessing IBAT inhibitory for use in combination with compounds of the present invention are disclosed in WO 03/106482

Suitable MAT inhibitors having the above structure for use in combination with compounds of the present invention are selected from any one of:
1,1-dioxo-3,3-dibutyl-5-phenyl-7-methylthio-8-(N—{(R)-α-[N′-((S)-1-carboxyethyl) carbamoyl]benzyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′4(S)-1-carboxypropyl) carbamoyl]benzyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′-((S)-1-carboxybutyl) carbamoyl]benzyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′-((S)-1-carboxy-2-methylpropyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′-((S)-1-carboxy-2-methylbutyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′-((S)-1-carboxy-3-methylbutyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′-((S)-1-carboxy-2-hydroxypropyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′-((S)-1-carboxy-2-mesylethyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′-((S)-1-carboxy-3-methylsulphonylpropyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′-((S)-1-carboxy-3-mesylpropyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′-((S)-1-carboxyethyl) carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′-((S)-1-carboxypropyl) carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′-((S)-1-carboxybutyl) carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′-((S)-1-carboxy-2-methylpropyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′-((S)-1-carboxy-2-methylbutyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′-((S)-1-carboxy-3-methylbutyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′-((S)-1-carboxy-2-hydroxyethyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′-((S)-1-carboxy-2-hydroxypropyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′-((S)-1-carboxy-2-methylthioethyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N—{(R)-[1-((S)-1-carboxy-2-methylsulphinylethyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′-((S)-1-carboxy-2-mesylethyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′-((S)-1-carboxy-2-methoxyethyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′-((S)-1-carboxy-3-methylthiopropyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′-(S)-1-carboxy-3-methylsulphonylpropyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′-((S)-1-carboxy-3-mesylpropyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′-((S)-1-carboxypropyl)carbamoyl]-4-hydroxybenzyl}carbamoylmethoxy)-2,3,4,5-tetrahydro-1,5-benzothiazepine; or
1,1-dioxo-3,3-dibutyl-5-phenyl-7-methylthio-8-(N—{(R)-α-[N′-((S)-1-carboxyethyl) carbamoyl]benzyl}carbamoylmethoxy)-2,3,4,5-tetrahydro-1,5-benzothiazepine.
   or a pharmaceutically acceptable salt, solvate, solvate of such a salt or a prodrug thereof.

Further suitable IBAT inhibitors for use in combination with compounds of the present invention are those disclosed in WO 04/076430.

In a particular aspect of the invention an IBAT inhibitor or a pharmaceutically acceptable salt, solvate, solvate of such a salt or a prodrug thereof is an IBAT inhibitor or a pharmaceutically acceptable salt thereof.

Therefore in an additional feature of the invention, there is provided a combination of a compound of formula (I), or a pharmaceutically acceptable salt, solvate, solvate of such a salt or a prodrug thereof and an IBAT inhibitor, or a pharmaceutically acceptable salt, solvate, solvate of such a salt or a prodrug thereof.

Therefore in an additional feature of the invention, there is provided a method for producing a cholesterol lowering effect in a warm-blooded animal, such as man, in need of such treatment which comprises administering to said animal an effective amount of a compound of formula (I), or a pharmaceutically acceptable salt, solvate, solvate of such a salt or a prodrug thereof in simultaneous, sequential or separate administration with an effective amount of an IBAT inhibitor, or a pharmaceutically acceptable salt, solvate, solvate of such a salt or a prodrug thereof.

According to a further aspect of the invention there is provided a pharmaceutical composition which comprises a compound of formula (I), or a pharmaceutically acceptable salt, solvate, solvate of such a salt or a prodrug thereof, and an IBAT inhibitor, or a pharmaceutically acceptable salt, solvate, solvate of such a salt or a prodrug thereof, in association with a pharmaceutically acceptable diluent or carrier.

According to a further aspect of the present invention there is provided a kit comprising a compound of formula (I), or a pharmaceutically acceptable salt, solvate, solvate of such a salt or a prodrug thereof, and an IBAT inhibitor, or a pharmaceutically acceptable salt, solvate, solvate of such a salt or a prodrug thereof.

According to a further aspect of the present invention there is provided a kit comprising:

a) a compound of formula (I), or a pharmaceutically acceptable salt, solvate, solvate of such a salt or a prodrug thereof, in a first unit dosage form;
b) an IBAT inhibitor, or a pharmaceutically acceptable salt, solvate, solvate of such a salt or a prodrug thereof; in a second unit dosage form; and
c) container means for containing said first and second dosage forms.

According to a further aspect of the present invention there is provided a kit comprising:

a) a compound of formula (I), or a pharmaceutically acceptable salt, solvate, solvate of such a salt or a prodrug thereof, together with a pharmaceutically acceptable diluent or carrier, in a first unit dosage form;
b) an IBAT inhibitor, or a pharmaceutically acceptable salt, solvate, solvate of such a salt or a prodrug thereof, in a second unit dosage form; and
c) container means for containing said first and second dosage forms.

According to another feature of the invention there is provided the use of a compound of the formula (I), or a pharmaceutically acceptable salt, solvate, solvate of such a salt or a prodrug thereof, and an IBAT inhibitor, or a pharmaceutically acceptable salt, solvate, solvate of such a salt or a prodrug thereof, in the manufacture of a medicament for use in the production of a cholesterol lowering effect in a warm-blooded animal, such as man.

According to a further aspect of the present invention there is provided a combination treatment comprising the administration of an effective amount of a compound of the formula (I), or a pharmaceutically acceptable salt, solvate, solvate of such a salt or a prodrug thereof, optionally together with a pharmaceutically acceptable diluent or carrier, with the simultaneous, sequential or separate administration of an effective amount of an IBAT inhibitor, or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

According to a further aspect of the present invention there is provided a combination treatment comprising the administration of an effective amount of a compound of the formula (I), or a pharmaceutically acceptable salt, solvate, solvate of such a salt or a prodrug thereof, optionally together with a pharmaceutically acceptable diluent or carrier, with the simultaneous, sequential or separate administration of an effective amount of an IBAT inhibitor, or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

In another aspect of the invention, the compound of formula (I), or a pharmaceutically acceptable salt, solvate, solvate of such a salt or a prodrug thereof, may be administered in association with a PPAR alpha and/or gamma and/or delta agonist, or pharmaceutically acceptable salts, solvates, solvates of such salts or prodrugs thereof. Suitable PPAR alpha and/or gamma and/or delta agonists, pharmaceutically acceptable salts, solvates, solvates of such salts or prodrugs thereof are well known in the art. These include the compounds described in WO 01/12187, WO 01/12612, WO 99/62870, WO 99/62872, WO 99/62871, WO 98/57941, WO 01/40170, WO 01/40172, WO 02/085844, WO 02/096863, WO03/051821, WO03/051822, WO03/051826, WO 04/000790, WO04/000295, WO04/000294, PCT/GB03/02584, PCT/GB03/02591, PCT/GB03/02598, J Med Chem, 1996, 39, 665, Expert Opinion on Therapeutic Patents, 10 (5), 623-634 (in particular the compounds described in the patent applications listed on page 634) and J Med Chem, 2000, 43, 527 which are all incorporated herein by reference. Particularly a PPAR alpha and/or gamma and/or delta agonist refers to muraglitazar (BMS 298585), rivoglitazone (CS-011), netoglitazone (MCC-555), balaglitazone (DRF-2593, N,N-2344), clofibrate, fenofibrate, bezafibrate, gemfibrozil, ciprofibrate, beclofibrate, etofibrate, gemcabene, pioglitazone, rosiglitazone, edaglitazone, LY-293111, IABX-2044, AVE-0847, AVE-8134, CLX-0921, DRF-10945, DRF-4832, LY-518674, naveglitazar (LY-818), LY-929, 641597, GW-590735, GW-677954, GW-501516, metaglidazen (MBX-102), T-131, SDX-101 E-3030, PLX-204, ONO-5129, KRP-101, R-483 (BM131258), TAK-559, K-111 (BM170744), netoglitazone (MCC-555; RWJ-241947; isaglitazone), FK-614 or TAK-654

For instance, a PPAR alpha and/or gamma and/or delta agonist refers to (S)-2-ethoxy-3-[4-(2-{4-methanesulphonyloxyphenyl}ethoxy)phenyl]propanoic acid (tesaglitazar) and pharmaceutically acceptable salts thereof.

Therefore in an additional feature of the invention, there is provided a combination of a compound of formula (I), or a pharmaceutically acceptable salt, solvate, solvate of such a salt or a prodrug thereof and a PPAR alpha and/or gamma agonist, or a pharmaceutically acceptable salt, solvate, solvate of such a salt or a prodrug thereof.

Therefore in an additional feature of the invention, there is provided a method for producing a cholesterol lowering effect in a warm-blooded animal, such as man, in need of such treatment which comprises administering to said animal an effective amount of a compound of formula (I), or a pharmaceutically acceptable salt, solvate, solvate of such a salt or a prodrug thereof in simultaneous, sequential or separate administration with an effective amount of a PPAR alpha and/or gamma and/or delta agonist, or a pharmaceutically acceptable salt, solvate, solvate of such a salt or a prodrug thereof.

According to a further aspect of the invention there is provided a pharmaceutical composition which comprises a compound of formula (I), or a pharmaceutically acceptable salt, solvate, solvate of such a salt or a prodrug thereof, and a PPAR alpha and/or gamma and/or delta agonist, or a pharmaceutically acceptable salt, solvate, solvate of such a salt or a prodrug thereof, in association with a pharmaceutically acceptable diluent or carrier.

According to a further aspect of the present invention there is provided a kit comprising a compound of formula (I), or a pharmaceutically acceptable salt, solvate, solvate of such a salt or a prodrug thereof, and a PPAR alpha and/or gamma and/or delta agonist, or a pharmaceutically acceptable salt, solvate, solvate of such a salt or a prodrug thereof.

According to a further aspect of the present invention there is provided a kit comprising:

a) a compound of formula (I), or a pharmaceutically acceptable salt, solvate, solvate of such a salt or a prodrug thereof, in a first unit dosage form;
b) a PPAR alpha and/or gamma and/or delta agonist, or a pharmaceutically acceptable salt, solvate, solvate of such a salt or a prodrug thereof; in a second unit dosage form; and
c) container means for containing said first and second dosage forms.

According to a further aspect of the present invention there is provided a kit comprising:

a) a compound of formula (I), or a pharmaceutically acceptable salt, solvate, solvate of such a salt or a prodrug thereof, together with a pharmaceutically acceptable diluent or carrier, in a first unit dosage form;
b) a PPAR alpha and/or gamma and/or delta agonist, or a pharmaceutically acceptable salt, solvate, solvate of such a salt or a prodrug thereof, in a second unit dosage form; and
c) container means for containing said first and second dosage forms.

According to another feature of the invention there is provided the use of a compound of the formula (I), or a pharmaceutically acceptable salt, solvate, solvate of such a salt or a prodrug thereof, and a PPAR alpha and/or gamma and/or delta agonist, or a pharmaceutically acceptable salt, solvate, solvate of such a salt or a prodrug thereof, in the manufacture of a medicament for use in producing a cholesterol lowering effect in a warm-blooded animal, such as man.

According to a further aspect of the present invention there is provided a combination treatment comprising the administration of an effective amount of a compound of the formula (I), or a pharmaceutically acceptable salt, solvate, solvate of such a salt or a prodrug thereof, optionally together with a pharmaceutically acceptable diluent or carrier, with the simultaneous, sequential or separate administration of an effective amount of a PPAR alpha and/or gamma and/or delta agonist, or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

In another aspect of the invention, there is provided a combination treatment comprising the administration of an effective amount of a compound of the formula (I), or a pharmaceutically acceptable salt, solvate, solvate of such a salt or a prodrug thereof, optionally together with a pharmaceutically acceptable diluent or carrier, with the simultaneous, sequential or separate administration of an -agonists to the receptor HM74A (nicotinic acid receptor). HM74A receptor agonists may be nicotine acid derivates. As used herein “nicotinic acid derivative” means a compounds comprising a pyridine-3-carboxylate structure or a pyrazine-2-carboxylate structure. Examples of nicotinic acid derivatives include nicotinic acid, niceritrol, nicofuranose, NIASPAN® and acipimox.

HM74A receptor agonists may be anthranilic acid derivatives described in WO-2005016867 and WO-2005016870.

Other nicotinic receptor agonists are for example compounds described in WO2005011677, WO2004032928 and WO2004033431.

Therefore, in an additional feature of the invention, there is provided a combination of a compound of formula (I), or a pharmaceutically acceptable salt, solvate, solvate of such a salt or a prodrug thereof and a HM74A receptor agonists or a pharmaceutically acceptable salt, solvate, solvate of such a salt or a prodrug thereof.

Therefore in an additional feature of the invention, there is provided a method for producing a cholesterol lowering effect in a warm-blooded animal, such as man, in need of such treatment which comprises administering to said animal an effective amount of a compound of formula (I), or a pharmaceutically acceptable salt, solvate, solvate of such a salt or a prodrug thereof in simultaneous, sequential or separate administration with an effective amount of a HM74A receptor agonists, or a pharmaceutically acceptable salt, solvate, solvate of such a salt or a prodrug thereof.

According to a further aspect of the invention there is provided a pharmaceutical composition which comprises a compound of formula (I), or a pharmaceutically acceptable salt, solvate, solvate of such a salt or a prodrug thereof, and a HM74A receptor agonists, or a pharmaceutically acceptable salt, solvate, solvate of such a salt or a prodrug thereof, in association with a pharmaceutically acceptable diluent or carrier.

In another aspect of the invention, there is provided a combination treatment comprising the administration of an effective amount of a compound of the formula (I), or a pharmaceutically acceptable salt, solvate, solvate of such a salt or a prodrug thereof, optionally together with a pharmaceutically acceptable diluent or carrier, with the simultaneous, sequential or separate administration of a mediator of reverse cholesterol transport i.e. a peptide (Apo A-1 mimetic peptides) or small molecule mediator of reverse cholesterol transport e.g. those described in Circ. 2002; 105:290, Circ. 2004. 109:3215, Curr. Opinion in Lipidology 2004, 15:645 or in WO2004094471.

In another aspect of the invention, the compound of formula I, or a pharmaceutically acceptable salt or solvate thereof, or a solvate of such a salt, may be administered in association with an anti-obesity compound, or pharmaceutically acceptable salts, solvates, solvates of such salts or prodrugs thereof, for example a pancreatic lipase inhibitor e.g. orlistat (EP 129,748) or an appetite (satiety) controlling substance for example sibutxamine (GB 2,184,122 and U.S. Pat. No. 4,929,629), a cannabinoid 1 (CB1) antagonist or inverse agonist, or pharmaceutically acceptable salts, solvates, solvates of such salts or prodrugs thereof, for example rimonabant (EP 656354) and as described in WO01/70700 or a melanin concentrating hormone (MCH) antagonist, or pharmaceutically acceptable salts, solvates, solvates of such salts or prodrugs thereof, for example as described in WO 04/004726.

According to another feature of the invention there is provided the use of a compound of the formula (I), or a pharmaceutically acceptable salt, solvate, solvate of such a salt or a prodrug thereof, and a nicotinic acid derivative, or a pharmaceutically acceptable salt, solvate, solvate of such a salt or a prodrug thereof, in the manufacture of a medicament for use in the production of a cholesterol lowering effect in a warm-blooded animal, such as man.

In another aspect of the invention, the compound of formula (I), or a pharmaceutically acceptable salt, solvate, solvate of such a salt or a prodrug thereof, may be administered in association with a bile acid sequestrant or a pharmaceutically acceptable salt, solvate, solvate of such a salt or a prodrug thereof. Suitable bile acid sequestrants include cholestyramine, cholestipol and cosevelam hydrochloride.

Therefore, in an additional feature of the invention, there is provided a combination of a compound of formula (I), or a pharmaceutically acceptable salt, solvate, solvate of such a salt or a prodrug thereof and a bile acid sequestrant or a pharmaceutically acceptable salt, solvate, solvate of such a salt or a prodrug thereof.

Therefore in an additional feature of the invention, there is provided a method for producing a cholesterol lowering effect in a warm-blooded animal, such as man, in need of such treatment which comprises administering to said animal an effective amount of a compound of formula (I), or a pharmaceutically acceptable salt, solvate, solvate of such a salt or a prodrug thereof in simultaneous, sequential or separate administration with an effective amount of a bile acid sequestrant, or a pharmaceutically acceptable salt, solvate, solvate of such a salt or a prodrug thereof.

According to a further aspect of the invention there is provided a pharmaceutical composition which comprises a compound of formula (I), or a pharmaceutically acceptable salt, solvate, solvate of such a salt or a prodrug thereof, and a bile acid sequestrant, or a pharmaceutically acceptable salt, solvate, solvate of such a salt or a prodrug thereof, in association with a pharmaceutically acceptable diluent or carrier.

According to another feature of the invention there is provided the use of a compound of the formula (I), or a pharmaceutically acceptable salt, solvate, solvate of such a salt or a prodrug thereof, and a bile acid sequestrant, or a pharmaceutically acceptable salt, solvate, solvate of such a salt or a prodrug thereof, in the manufacture of a medicament for use in the production of a cholesterol lowering effect in a warm-blooded animal, such as man.

In another aspect of the invention, the compound of formula I, or a pharmaceutically acceptable salt or solvate thereof, or a solvate of such a salt, may be administered in association with a cholesteryl ester transfer protein (CETP) inhibitor, or pharmaceutically acceptable salts, solvates, solvates of such salts or prodrugs thereof, for example ITT-705, torcetrapib (CP-529414), Bay 194789 and those referenced and described in WO05033082 or WO 00/38725 page 7 line 22—page 10, line 17 which are incorporated herein by reference.

In another aspect of the invention, the compound of formula I, or a pharmaceutically acceptable salt or solvate thereof, or a solvate of such a salt, may be administered in association with a acyl coenzymA: cholesterol O-acyltransferase (ACAT) inhibitor, or pharmaceutically acceptable salts, solvates, solvates of such salts or prodrugs thereof, for example pactimibe (CS-505), eflucimibe (F-12511) and SMP-797, avasimibe or K604.

In yet another aspect of the invention, the compound of formula I, association with modulators for example GW-4064 and INT-747 of nuclear receptors such as farnesoid or a pharmaceutically acceptable salt or solvate thereof, or a solvate of such a salt, may be administered in X receptor (FXR), or pharmaceutically acceptable salt, solvate, solvate of such a salt or a prodrug thereof.

In another aspect of the invention, the compound of formula I, or a pharmaceutically acceptable salt or solvate thereof, or a solvate of such a salt, may be administered in association with a phytosterol compound, or pharmaceutically acceptable salts, solvates, solvates of such salts or prodrugs thereof, for example stanols. An example of phytosterol analogs is FM-VP4.

In another aspect of the invention, the compound of formula I, or a pharmaceutically acceptable salt or solvate thereof, or a solvate of such a salt, may be administered in association with other therapies for the treatment of metabolic syndrome or type 2 diabetes and its associated complications, these include biguanide drugs, for example metformin, phenformin and buformin, insulin (synthetic insulin analogues, amylin) and oral antihyperglycemics (these are divided into prandial glucose regulators and alpha-glucosidase inhibitors). An example of an alpha-glucosidase inhibitor is acarbose or voglibose or miglitol. An example of a prandial glucose regulator is repaglinide or nateglinide.

In another aspect of the invention, the compound of formula I, or a pharmaceutically acceptable salt or solvate thereof, or a solvate of such a salt, may be administered in association with a sulfonylurea for example: glimepiride, glibenclamide (glyburide), gliclazide, glipizide, gliquidone, chloropropamide, tolbutamide, acetohexamide, glycopyramide, carbutamide, glibonuride, glisoxepid, glybuthiazole, glibuzole, glyhexamide, glymidine, glypinamide, phenbutamide, tolcylamide and tolazamide. Preferably the sulfonylurea is glimepiride or glibenclamide (glyburide). More preferably the sulfonylurea is glimepiride. Therefore the present invention includes administration of a compound of the present invention in conjunction with one, two or more existing therapies described in this paragraph. The doses of the other existing therapies for the treatment of type 2 diabetes and its associated complications will be those known in the art and approved for use by regulatory bodies for example the FDA and may be found in the Orange Book published by the FDA. Alternatively smaller doses may be used as a result of the benefits derived from the combination.

According to an additional further aspect of the present invention there is provided a combination treatment comprising the administration of an effective amount of a compound of the formula (I), or a pharmaceutically acceptable salt, solvate, solvate of such a salt or a prodrug thereof, optionally together with a pharmaceutically acceptable diluent or carrier, with the simultaneous, sequential or separate administration one or more of the following agents selected from Group X:
an antihypertensive compound (for example althiazide, benzthiazide, captopril, carvedilol, chlorothiazide sodium, clonidine hydrochloride, cyclothiazide, delapril hydrochloride, dilevalol hydrochloride, doxazosin mesylate, fosinopril sodium, guanfacine hydrochloride, methyldopa, metoprolol succinate, moexipril hydrochloride, monatepil maleate, pelanserin hydrochloride, phenoxybenzemine hydrochloride, prazosin hydrochloride, primidolol, quinapril hydrochloride, quinaprilat, ramipril, terazosin hydrochloride, candesartan, candesartan cilexetil, telmisartan, amlodipine besylate, amlodipine maleate and bevantolol hydrochloride);
an angiotensin converting enzyme inhibitor (for example alacepril, alatriopril, altiopril calcium, ancovenin, benazepril, benazepril hydrochloride, benazeprilat, benzoylcaptopril, captopril, captopril-cysteine, captopril-glutathione, ceranapril, ceranopril, ceronapril, cilazapril, cilazaprilat, delapril, delapril-diacid, enalapril, enalaprilat, enapril, epicaptopril, foroxymithine, fosfenopril, fosenopril, fosenopril sodium, fosinopril, fosinopril sodium, fosinoprilat, fosinoprilic acid, glycopril, hemorphin-4, idrapril, imidapril, indolapril, indolaprilat, libenzapril, lisinopril, lyciumin A, lyciumin B, mixanpril, moexipril, moexiprilat, moveltipril, muracein A, muracein B, muracein C, pentopril, perindopril, perindoprilat, pivalopril, pivopril, quinapril, quinapril hydrochloride, quinaprilat, ramipril, ramiprilat, spirapril, spirapril hydrochloride, spiraprilat, spiropril, spiropril hydrochloride, temocapril, temocapril hydrochloride, teprotide, trandolapril, trandolaprilat, utibapril, zabicipril, zabiciprilat, zofenopril and zofenoprilat);
an angiotensin II receptor antagonist (for example candesartan, candesartan cilexetil, losartan, valsartan, irbesartan, tasosartan, telmisartan and eprosartan);
an andrenergic blocker (for example bretylium tosylate, dihydroergotamine so mesylate, phentolamine mesylate, solypertine tartrate, zolertine hydrochloride, carvedilol or labetalol hydrochloride); an alpha andrenergic blocker (for example fenspiride hydrochloride, labetalol hydrochloride, proroxan and alfuzosin hydrochloride); a beta andrenergic blocker (for example acebutolol, acebutolol hydrochloride, alprenolol hydrochloride, atenolol, bunolol hydrochloride, carteolol hydrochloride, celiprolol hydrochloride, cetamolol hydrochloride, cicloprolol hydrochloride, dexpropranolol hydrochloride, diacetolol hydrochloride, dilevalol hydrochloride, esmolol hydrochloride, exaprolol hydrochloride, flestolol sulfate, labetalol hydrochloride, levobetaxolol hydrochloride, levobunolol hydrochloride, metalol hydrochloride, metoprolol, metoprolol tartrate, nadolol, pamatolol sulfate, penbutolol sulfate, practolol, propranolol hydrochloride, sotalol hydrochloride, timolol, timolol maleate, tiprenolol hydrochloride, tolamolol, bisoprolol, bisoprolol fumarate and nebivolol); or a mixed alpha/beta andrenergic blocker;
an andrenergic stimulant (for example combination product of chlorothiazide and methyldopa, the combination product of methyldopa hydrochlorothiazide and methyldopa, clonidine hydrochloride, clonidine, the combination product of chlorthalidone and clonidine hydrochloride and guanfacine hydrochloride);
channel blocker, for example a calcium channel blocker (for example clentiazem maleate, amlodipine besylate, isradipine, nimodipine, felodipine, nilvadipine, nifedipine, teludipine hydrochloride, diltiazem hydrochloride, belfosdil, verapamil hydrochloride or fostedil);
a diuretic (for example the combination product of hydrochlorothiazide and spironolactone and the combination product of hydrochlorothiazide and triamterene);
anti-anginal agents (for example amlodipine besylate, amlodipine maleate, betaxolol hydrochloride, bevantolol hydrochloride, butoprozine hydrochloride, carvedilol, cinepazet maleate, metoprolol succinate, molsidomine, monatepil maleate, primidolol, ranolazine hydrochloride, tosifen or verapamil hydrochloride);
vasodilators for example coronary vasodilators (for example fostedil, azaclorzine hydrochloride, chromonar hydrochloride, clonitrate, diltiazem hydrochloride, dipyridamole, droprenilamine, erythrityl tetranitrate, isosorbide dinitrate, isosorbide mononitrate, lidoflazine, inioflazine hydrochloride, mixidine, molsidomine, nicorandil, nifedipine, nisoldipine, nitroglycerine, oxprenolol hydrochloride, pentrinitrol, perhexyline maleate, prenylamine, propatyl nitrate, terodiline hydrochloride, tolamolol and veraparnil);
anti-coagulants (selected from argatroban, bivalirudin, dalteparin sodium, desirudin, dicumarol, Iyapolate sodium, nafamostat mesylate, phenprocoumon, tinzaparin sodium and warfarin sodium);
antithrombotic agents (for example anagrelide hydrochloride, bivalirudin, cilostazol, dalteparin sodium, danaparoid sodium, dazoxiben hydrochloride, efegatran sulfate, enoxaparin sodium, fluretofen, ifetroban, ifetroban sodium, lamifiban, lotrafiban hydrochloride, napsagatran, orbofiban acetate, roxifiban acetate, sibrafiban, tinzaparin sodium, trifenagrel, abciximab and zolimomab aritox);
fibrinogen receptor antagonists (for example roxifiban acetate, fradafiban, orbofiban, lotrafiban hydrochloride, tirofiban, xemilofiban, monoclonal antibody 7E3 and sibrafiban)
platelet inhibitors (for example cilostezol, clopidogrel bisulfate, epoprostenol, epoprostenol sodium, ticlopidine hydrochloride, aspirin, ibuprofen, naproxen, sulindae, indomethacin, mefenamate, droxicam, diclofenac, sulfinpyrazone and piroxicam, dipyridamole);
platelet aggregation inhibitors (for example acadesine, beraprost, beraprost sodium, ciprostene calcium, itezigrel, lifarizine, lotrafiban hydrochloride, orbofiban acetate, oxagrelate, fradafiban, orbofiban, tirofiban and xemilofiban)
hemorrheologic agents (for example pentoxifylline);
lipoprotein associated coagulation inhibitors;
Factor Vila inhibitors;
A Factor Xa inhibitors;
A low molecular weight heparins (for example enoxaparin, nardroparin, dalteparin, certroparin, parnaparin, reviparin and tinzaparin);
liver X receptor (LXR) agonists for example GW-3965 and those described in WO00224632, WO00103705, WO02090375 and WO00054759 (claim 1 and the named examples of these four application are incorporated herein by reference);
microsomal triglyceride transfer protein inhibitors for example implitapide, CP-346086, JTT-130, BMS-201038, R-103757 and those described in WO05/021486, WO03004020, WO03002533, WO02083658 and WO 00242291 (claim 1 and the named examples of these four application are incorporated herein by reference);

ApoA1 expression inducer for example those described in WO2005032559 or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

Therefore, in an additional feature of the invention, there is provided a combination of a compound of formula (I), or a pharmaceutically acceptable salt, solvate, solvate of such a salt or a prodrug thereof and a compound from Group X or a pharmaceutically acceptable salt, solvate, solvate of such a salt or a prodrug thereof.

Therefore in an additional feature of the invention, there is provided a method for producing a cholesterol lowering effect in a warm-blooded animal, such as man, in need of such treatment which comprises administering to said animal an effective amount of a compound of formula (I), or a pharmaceutically acceptable salt, solvate, solvate of such a salt or a prodrug thereof in simultaneous, sequential or separate administration with an effective amount of a compound from Group X, or a pharmaceutically acceptable salt, solvate, solvate of such a salt or a prodrug thereof.

According to a further aspect of the invention there is provided a pharmaceutical composition which comprises a compound of formula (I), or a pharmaceutically acceptable salt, solvate, solvate of such a salt or a prodrug thereof, and a compound from Group X, or a pharmaceutically acceptable salt, solvate, solvate of such a salt or a prodrug thereof, in association with a pharmaceutically acceptable diluent or carrier.

According to another feature of the invention there is provided the use of a compound of the formula (I), or a pharmaceutically acceptable salt, solvate, solvate of such a salt or a prodrug thereof, and a compound from Group X, or a pharmaceutically acceptable salt, solvate, solvate of such a salt or a prodrug thereof, in the manufacture of a medicament for use in the production of a cholesterol lowering effect in a warm-blooded animal, such as man.

In addition to their use in therapeutic medicine, the compounds of formula (I), or a pharmaceutically acceptable salt, solvate, solvate of such a salt or a prodrug thereof, are also useful as pharmacological tools in the development and standardisation of in vitro and in vivo test systems for the evaluation of the effects of inhibitors of cholesterol absorption in laboratory animals such as cats, dogs, rabbits, monkeys, rats and mice, as part of the search for new therapeutic agents.

In the above other pharmaceutical composition, process, method, use and medicament manufacture features, the alternative and preferred embodiments of the compounds of the invention described herein also apply.

### EXAMPLES

The invention will now be illustrated in the following non limiting Examples, in which standard techniques known to the skilled chemist and techniques analogous to those described in these Examples may be used where appropriate, and in which, unless otherwise stated:

(i) evaporations were carried out by rotary evaporation in vacuo and work up procedures were carried out after removal of residual solids such as drying agents by filtration;
(ii) all reactions were carried out under an inert atmosphere at ambient temperature, typically in the range 18-25° C., with solvents of HPLC grade under anhydrous conditions, unless otherwise stated;
(iii) column chromatography (by the flash procedure) was performed on Silica gel 40-63 μm (Merck);
(iv) yields are given for illustration only and are not necessarily the maximum attainable;
(v) the structures of the end products of the formula (I) were generally confirmed by nuclear (generally proton) magnetic resonance (NMR) and mass spectral techniques; magnetic resonance chemical shift values were measured in deuterated CDCl₃ (unless otherwise stated) on the delta scale (ppm downfield from tetramethylsilane); proton data is quoted unless otherwise stated; spectra were recorded on a Varian Mercury-300 MHz, Varian Unity plus-400 MHz, Varian Unity plus-600 MHz or on Varian Inova-500 MHz spectrometer unless otherwise stated data was recorded at 400 MHz; and peak multiplicities are shown as follows: s, singlet; d, doublet; dd, double doublet; t, triplet; tt, triple triplet; q, quartet; tq, triple quartet; m, multiplet; br, broad; ABq, AB quartet; ABd, AB doublet, ABdd, AB doublet of doublets; dABq, doublet of AB quartets;

Mass spectra were recorded on one of the following instruments: LCT, QTOF, ZQ Mass spectrometer, all from Waters.

### LC-MS:

Separation was performed using Agilent 1100 Series Modules or Waters 1525 pump on a Synergi MAX-RP (Phenomenex) C12 3×50 mm 4 μm with gradient elution.

Samples were injected using Waters 2700 Sample Manager.

### Mobile Phases:

Generic gradients were applied from 5% to 95% acetonitrile.

Buffers containing 10 mM ammonium acetate or 5 mM ammonium formiate/5 mM formic acid were used.

The mass spectra were recorded with a Waters ZQ2000 or Waters ZMD equipped with an electrospray interface, switching positive and negative ionization mode. UV spectra were collected by a Aglent 1100 PDA or Waters 2996 DAD and the evaporative light scattering (ELS) signal by a Sedere Sedex 55 or 75.

Data collection and evaluation were performed using the MassLynx software. Accurate mass data were determined using either a LCT or QTOF MS (Waters) with leucine enkephaline (m/z 556.2771) as lockmass. Unless otherwise stated the mass ion quoted is (MH⁺).

Unless further details are specified in the text, analytical high performance liquid chromatography (HPLC) was performed on Prep LC 2000 (Waters), Cromasil C₈, 7 μm, (Akzo Nobel); MeCN and de-ionised water 10 mM ammonium acetate as mobile phases, with suitable composition;

(vii) intermediates were not generally fully characterised and purity was assessed by thin layer chromatography (TLC), HPLC, infra-red (IR), MS or NMR analysis;
(viii) where solutions were dried sodium sulphate was the drying agent; and
(ix) the following abbreviations may be used hereinbefore or hereinafter:—
DCM dichloromethane;

### DMF N,N-dimethylformamide;

TBTU o-Benzotriazol-1-yl-N,N,N′,N′-tetramethyluronium tetrafluoroborate;
EtOAc ethyl acetate;
MeCN acetonitrile;
TFA trifluoroacetic acid;
DMAP 4-(dimethylamino)pyridine;

### BSA N,O-Bis(trimethylsilyl)acetamide; and

TBAF tetrabutylammonium fluoride;
NMM N-methyl morpholine;
TEA triethylamine;
DBN 1,5-diazabicyclo-[4,3,0]-non-5-ene.

### Examples

### Example 1

### N-{(2R)-2-[(N-{[4-((2R,3R)-1-(4-fluorophenyl)-3-{[2-(4-fluorophenyl)-2-hydroxyethyl]thio}-4-oxoazetidin-2-yl)phenoxy]acetyl}glycyl)amino]-2-phenylacetyl}glycine

(2R)-[(N-{[4-((2R,3R)-1-(4-fluorophenyl)-3-{[2-(4-fluorophenyl)-2-hydroxyethyl]thio}-4-oxoazetidin-2-yl)phenoxy]acetyl}glycyl)amino](phenyl)acetic acid (19 mg, 0.028 mmol) glycine-ter-butyl ester (10 mg, 0.076 mmol), N-methylmorpholine (9 ul, 0.084 mmol) and TBTU (14 mg, 0.042 mmol) were added to methylene chloride (2 ml) and the mixture was stirred at room temperature for 2 h. The solvent was evaporated under reduced pressure. To the residue was added formic acid (1 ml) and the reaction mixture was stirred at room temperature for 4 h. The solvent were evaporated under reduced pressure and co-evaporated with toluene. To the residue were added methanol (1.5 ml) and triethylamine (0.3 ml) and the mixture was stirred for 15 h and the solvent were evaporated under reduced pressure. The residue was purified by preparative HPLC using acetonitril/ammonium acetate buffer (45:55) as eluent. The collected fractions were lyophilized to obtain the title compound.

(¹H-NMR, 400 MHz, CD₃OD): 2.9-3.1 (m, 2H), 3.85 (ABq, 2H), 4.0-4.1 (m, 3H), 4.6 (s, 2H), 4.8-4.95 (m, 2H), 5.55 (s, 1H), 6.95-7.1 (m, 6H), 7.15-7.4 (m, 9H), 7.45-7.5 (m, 2H)

### Example 2

### N-{[4-((2R,3R)-1-(4-fluorophenyl)-3-{[2-(4-fluorophenyl)-2-hydroxyethyl]thio}-4-oxoazetidin-2-yl)phenoxy]acetyl}glycylglycyl-N-benzylglycine

To a solution of N-{[4-((2R,3R)-1-(4-fluorophenyl)-3-{[2-(4-fluorophenyl)-2-oxoethyl]thio}-4-oxoazetidin-2-yl)phenoxy]acetyl}glycylglycine (0.020 g, 0.033 mmol) and NMM (0.015 ml, 0.136 mmol) in DMF (2 ml) at 30° C. was added TBTU (0.015 g, 0.047 mmol). After 30 min was N-benzylglycine (0.006 g, 0.036 mmol, 98%) added and the mixture was stirred at 30° C. for 2 h 30 min. The reaction was quenched by the addition of water (1 ml) and the resulting mixture was diluted with MeOH (2 ml). To this solution was added NaBH4 (0.020 g, 0.529 mmol) and the mixture was stirred for 10 min. The reaction was quenched by the addition of a 0.1M ammonium acetate buffer (3 ml) before most of the methanol was removed under reduced pressure. The remaining solution was purified by preparative HPLC, using a gradient of 20-50% MeCN in 0.1M ammonium acetate buffer as eluent. Freeze-drying of the pure fractions gave the title compound.

Accurate mass: 747.2315 (M+1)⁺

### Example 3

### N-{[4-((2R,3R)-1-(4-fluorophenyl)-3-{[2-(4-fluorophenyl)-2-hydroxyethyl]thio}-4-oxoazetidin-2-yl)phenoxy]acetyl}glycylglycyl-N-ethylglycine

To a solution of N-{[4-((2R,3R)-1-(4-fluorophenyl)-3-{[2-(4-fluorophenyl)-2-oxoethyl]thio}-4-oxoazetidin-2-yl)phenoxy]acetyl}glycylglycine (0.020 g, 0.033 mmol) and NMM (0.015 ml, 0.136 mmol) in DMF (2 ml) at 30° C. was added TBTU (0.015 g, 0.047 mmol). After 30 min was N-ethylglycine (0.004 g, 0.038 mmol, 98%) added and the mixture was stirred at 30° C. for 2 h. The reaction was quenched by the addition of water (1 ml) and the resulting mixture was diluted with MeOH (2 ml). NaBH4 (0.020 g, 0.529 mmol) was added and the mixture was stirred for 10 min. The reaction was quenched by the addition of a 0.1M ammonium acetate buffer (3 ml) before most of the methanol was removed under reduced pressure. The remaining solution was purified twice by preparative HPLC; first by using a gradient of 20-50% MeCN in a 0.1M ammonium acetate buffer as eluent and then with 40% MeCN in a 0.1M ammonium acetate buffer as eluent. Freeze-drying of the pure fractions gave the title compound.

Accurate mass: 685.2147 (M+1)⁺

### Example 4

### N-{[4-((2R,3R)-1-(4-fluorophenyl)-3-{[2-(4-fluorophenyl)-2-hydroxyethyl]thio}-4-oxoazetidin-2-yl)phenoxy]acetyl}glycylglycyl-3-methyl-D-valine

To a solution of N-{[4-((2R,3R)-1-(4-fluorophenyl)-3-{[2-(4-fluorophenyl)-2-oxoethyl]thio}-4-oxoazetidin-2-yl)phenoxy]acetyl}glycylglycine (0.020 g, 0.033 mmol) and NMM (0.015 ml, 0.136 mmol) in DMF (2 ml) at 30° C. was added TBTU (0.015 g, 0.047 mmol). After 30 min was D-tert-leucine (0.004 g, 0.034 mmol) added and the mixture was stirred at 30° C. for 21 h. The reaction was quenched by the addition of water (1 ml) and the resulting mixture was diluted with MeOH (2 ml). NaBH4 (0.015 g, 0.397 mmol) was added and the mixture was stirred for 10 min. The reaction was quenched by the addition of a 0.1M ammonium acetate buffer (3 ml) before most of the methanol was removed under reduced pressure. The remaining solution was purified by preparative HPLC using 40% MeCN in a 0.1M ammonium acetate buffer as eluent. Freeze-drying of the pure fractions gave the title compound.

ES− M/z: 711.1 (M−1)⁻. ¹H NMR (DMSO, 400 MHz): 0.90 (s, 9H), 2.85-2.95 (m, 2H), 3.74-3.80 (m, 4H), 3.98-4.04 (m, 1H), 4.25-4.30 (m, 1H), 4.53 (s, 2H), 4.69-4.77 (m, 1H), 5.04-5.09 (m, 1H), 6.95-7.41 (m, 12H), 7.68-7.75 (m, 1H), 8.11-8.17 (m, 1H), 8.31-8.37 (m, 1H).

### Example 5

### N-{[4-((2R,3R)-1-(4-fluorophenyl)-3-{[(2R or S)-2-(4-fluorophenyl)-2-hydroxyethyl]thio}-4-oxoazetidin-2-yl)phenoxy]acetyl}glycyl-D-lysyl-D-lysine acetate

A mixture of N-{[4-((2R,3R)-1-(4-fluorophenyl)-3-{[(2R or S)-2-(4-fluorophenyl)-2-hydroxyethyl]thio}-4-oxoazetidin-2-yl)phenoxy]acetyl}glycine (AR-H81728, 0.0093 g, 0.017 mmole), N-methylmorpholin (0.010 mL, 0.051 mmole) in DMF (1 mL) was stirred, TBTU (0.0077 g, 0.025 mmole) was added. The mixture was stirred for 50 minutes under N₂-atmosphere. N⁶-[(9H-fluoren-9-ylmethoxy)carbonyl]-D-lysine (0.010 g, 0.025 mmole) was added and stirred for 1 hour. A small amount of water was added and the solvent was removed under reduced pressure. The residue was purified by preparative HPLC on a Kromasil C8-column using a gradient of 5-100% MeCN in 0.1M ammonium acid buffer as eluent. After removing the solvent under reduced pressure and freeze-drying from water, the residue was dissolved in 0.5 mL of piperidine in DMF (23% by volume). After about ten minutes the solvent was removed under reduced pressure and the residue was purified by preparative HPLC on a Kromasil C8-column using a gradient of 5-100% MeCN in 0.1M ammonium acid buffer as eluent. After removing the solvent under reduced pressure and freeze-drying from water, the title compound was obtained.

NMR (500 MHz, CD3COOD) 1.36-1.58 (m, 4H), 1.58-1.81 (m, 6H), 1.82-1.95 (m, 5H), 2.88-2.95 (m, 4H), 2.98-3.09 (m, 2H), 3.94-4.03 (m, 3H), 4.26 (dd, 1H), 4.39 (t, 1H), 4.62 (s, 2H), 4.81-4.87 (m, 1H), 4.93 (d, 1H), 6.98-7.11 (m, 6H), 7.27-7.33 (m, 2H), 7.33-7.40 (m, 4H)

### Example 6

### N-{[4-((2R,3R)-1-(4-fluorophenyl)-3-{[(2R or S)-2-(4-fluorophenyl)-2-hydroxyethyl]thio}-4-oxoazetidin-2-yl)phenoxy]acetyl}glycyl-3-methyl-D-valylglycine

To a stirred solution of N-{[4-((2R,3R)-1-(4-fluorophenyl)-3-{[(2R or S)-2-(4-fluorophenyl)-2-hydroxyethyl]thio}-4-oxoazetidin-2-yl)phenoxy]acetyl}glycyl-3-methyl-D- (16.9 mg, 0.026 mmol) in DCM (3 ml) were added 1-Ethyl-3-(3-dimethylaminopropyl)carbodiimide hydrochloride, EDC, (6.8 mg, 0.035 mmol) and tert-butyl glycinate hydrochloride (5.4 mg, 0.032 mmol). N,N-dimethylpyridin-4-amine, DMAP, (1.6 mg, 0.013 mmol) was added and the reaction mixture was stirred overnight. The formation of the tert-butyl ester of the title compound was confirmed. M/z: 767.37(M−1). The solvent was removed under reduced pressure. The residue was dissolved in formic acid (3 ml) and stirred for 2 hours. Analysis with LC-MS showed the hydrolysed product but with a formyl-group on the alcohol. The formic acid was co-evaporated with toluene. The residue was dissolved in methanol (3 ml) and Et₃N (200 μl, 1.44 mmol) was added and the mixture was stirred for 1 hour. The solvent was removed under reduced pressure and the residue was purified with preparative HPLC on a C8 column. A gradient from 20 to 60% MeCN in 0.1M NH₄OAc buffer was used as eluent. The MeCN was removed under reduced pressure and the remaining water solution was diluted with DMC. The water phase was acidified with KHSO₄ (2M) to pH 2. The phases were separated and the organic phase was passed through a phase separator. The solvent was removed under reduced pressure and the residue was dissolved in MeCN and water. After lyophilisation, the title compound was obtained.

H-NMR (500 MHz, DMSO-d₆): 1.31 (s, 9H), 3.35 (d, 2H), 3.98 (t, 2H), 4.25 (d, 2H), 4:67 (d, 1H), 4.69 (d, 1H), 4.95 (s, 2H), 5.14 (t, 1H), 5.50 (d, 1H), 7.41 (d, 2H), 7.50-7.60 (m, 4H), 7.64-7.69 (m, 2H), 7.74-7.81 (m, 4H), 8.26 (d, 1H), 8.39 (b, 1H), 8.79 (b, 1H). M/z: 711.32 (M−1).

### Example 7

### N-{[4-((2R,3R)-1-(4-fluorophenyl)-3-{[(2R or S)-2-(4-fluorophenyl)-2-hydroxyethyl]thio}-4-oxoazetidin-2-yl)phenoxy]acetyl}glycyl-3-methyl-D-valyl-D-serine

To a stirred solution of N-{[4-((2R,3R)-1-(4-fluorophenyl)-3-{[(2R or S)-2-(4-fluorophenyl)-2-hydroxyethyl]thio}-4-oxoazetidin-2-yl)phenoxy]acetyl}glycyl-3-methyl-D-valine and tert-butyl O-(tert-butyl)-D-serinate hydrochloride (7.5 mg, 0.030 mmol) was added N-methylmorpholine (10 in, 0.090 mmol). TBTU (10.8 mg, 0.034 mmol) was added and the reaction mixture was stirred overnight. The formation of the intermediate, the tert-butyl protected serine compound, was confirmed. M/z: 856 (M−1) and 854 (M−1). The solvent was removed under reduced pressure. The residue was dissolved in formic acid (5 ml) and the mixture was stirred for 4 hours. Analysis with LC-MS showed the hydrolysed product with formyl-groups on both alcohol functions. The formic acid was co-evaporated with toluene. The residue was dissolved in methanol (3 ml) and Et₃N (200 μl, 1.44 mmol) was added. The reaction mixture was stirred for 2 hours. The solvent was removed under reduced pressure and the residue was purified with preparative HPLC on a C8 column. A gradient from 20 to 55% MeCN in 0.1M NH₄OAc buffer was used as eluent. The MeCN was removed under reduced pressure and the remaining water solution was diluted with additional water and DCM. The solution was acidified with KHSO₄ (2M) and extracted. The organic phase was passed through a phase separator and the solvent was removed under reduced pressure. The residue was dissolved in MeCN and water. After lyophilisation, the title compound was obtained

H-NMR (500 MHz, DMSO-d₆): 0.89 (s, 9H), 2.92 (d, 2H), 3.53-3.67 (m, 2H), 3.81-3.85 (m, 2H), 4.13 (b, 1H), 4.26 (d, 1H), 4.33 (d, 1H), 4.52 (s, 2H), 4.71 (b, 1H), 5.07 (d, 1H), 5.65 (bd, 1H), 6.99 (d, 2H), 7.08-7.18 (m, 4H), 7.21-7.26 (m, 2H), 7.32-7.40 (m, 4H), 7.80 (d, 1H), 8.09 (b, 1H), 8.29 (t, 1H). M/z: 741.52 (M−1).

### Example 8

### N-{[4-((2R,3R)-1-(4-fluorophenyl)-3-{[(2R or S)-2-(4-fluorophenyl)-2-hydroxyethyl]thio}-4-oxoazetidin-2-yl)phenoxy]acetyl}glycyl-3-cyclohexyl-D-alanylglycine

To a solution of N-{[4-((2R,3R)-1-(4-fluorophenyl)-3-{[(2R or S)-2-(4-fluorophenyl)-2-hydroxyethyl]thio}-4-oxoazetidin-2-yl)phenoxy]acetyl}glycyl-3-cyclohexyl-D-alanine (0.025 g, 0.036 mmol) in DMF (2 ml) was added 3,4-dichlorophenol (0.007 g, 0.040 mmol) followed by the addition of N-methylmorpholine (0.009 g, 0.09 mmol) and TBTU (0.0115 g, 0.036 mmol). After 6 h of stirring at room temperature, full conversion to the corresponding 3,4-dichlorophenylester intermediate (3,4-dichlorophenyl N-{[4-(2R,3R)-1-(4-fluorophenyl)-3-{[(2R)-2-(4-fluorophenyl)-2-hydroxyethyl]thio}-4-oxoazetidin-2-yl)phenoxy]acetyl}glycyl-3-cyclohexyl-D-alaninate) had been achieved. To this intermediate, was added glycine (0.003 g, 0.046 mmol) followed by the addition of lithium chloride (0.03 g, 0.71 mmol). The mixture was allowed to stir over night. Preparative HPLC of the mixture using an eluent of 10-50% CH₃CN in 0.1M NH₄OAc buffer afforded, after freeze drying of pure fractions, the titled compound was obtained.

¹H NMR [(CD₃)₂SO), 400 MHz] δ 0.74-1.65 (m, 13H), 2.89 (d, 2H), 3.54 (d, 2H), 3.76 (d, 2H), 4.23 (d, 1H), 4.26-4.33 (m, 1H), 4.49 (s, 2H), 4.68 (t, 1H), 5.04 (d, 1H), 6.95-7.35 (m, 12H), 7.86-7.94 (m, 1H), 8.05 (d, 1H), 8.22 (t, 1H).

### Example 9

### N-{[4-((2R,3R)-1-(4-fluorophenyl)-3-{[(2R or S)-2-(4-fluorophenyl)-2-hydroxyethyl]thio}-4-oxoazetidin-2-yl)phenoxy]acetyl}glycyl-3-cyclohexyl-D-alanyl-D-alanine

The titled compound was prepared using the same procedure as that used for the synthesis of N-{[4-((2R,3R)-1-(4-fluorophenyl)-3-{[(2R or S)-2-(4-fluorophenyl)-2-hydroxyethyl]thio}-4-oxoazetidin-2-yl)phenoxy]acetyl}glycyl-3-cyclohexyl-D-alanylglycine but using D-alanine instead of glycine.

¹H NMR [(CD₃)₂SO), 400 MHz] δ 0.73-1.67 (m, 16H), 2.89 (d, 2H), 3.75 (d, 2H), 3.86-3.94 (m, 1H), 4.23 (d, 1H), 4.24-4.31 (m, 1H), 4.49 (s, 2H), 4.68 (t, 1H), 5.04 (d, 1H), 6.95-7.35 (m, 12H), 7.78-7.87 (m, 1H), 8.03-8.09 (m, 1H), 8.22-8.32 (m, 1H).

### Example 10

### N-{[4-((2R,3R)-1-(4-fluorophenyl)-3-{[(2R or S)-2-(4-fluorophenyl)-2-hydroxyethyl]thio}-4-oxoazetidin-2-yl)phenoxy]acetyl}glycyl-3-cyclohexyl-D-alanyl-D-asparagine

The titled compound was prepared using the same procedure as that used for the synthesis of N-{[4-((2R,3R)-1-(4-fluorophenyl)-3-{[(2R or S)-2-(4-fluorophenyl)-2-hydroxyethyl]thio}-4-oxoazetidin-2-yl)phenoxy]acetyl}glycyl-3-cyclohexyl-D-alanylglycine but using D-asparagine instead of glycine.

¹H NMR [(CD₃)₂SO), 400 MHz] δ 0.74-1.67 (m, 13H), 2.24-2.46 (m, 2H), 2.89 (d, 2H), 3.69-3.82 (m, 2H), 4.11-4.18 (m, 1H), 4.23 (d, 1H), 4.23-4.31 (m, 1H), 4.49 (s, 2H), 4.68 (t, 1H), 5.04 (d, 1H), 6.67-6.75 (m, 1H), 6.95-7.35 (m, 12H), 7.74-7.87 (m, 8.06-8.10 (m, 1H), 8.21-8.27 (m, 1H).

### Example 11

### N-{[4-((2R,3R)-1-(4-fluorophenyl)-3-{[(2R or S)-2-(4-fluorophenyl)-2-hydroxyethyl]thio}-4-oxoazetidin-2-yl)phenoxy]acetyl}glycyl-3-cyclohexyl-D-alanyl-D-phenylalanine

The titled compound was prepared using the same procedure as that used for the synthesis of N-{[4-((2R,3R)-1-(4-fluorophenyl)-3-{[(2R or S)-2-(4-fluorophenyl)-2-hydroxyethyl]thio}-4-oxoazetidin-2-yl)phenoxy]acetyl}glycyl-3-cyclohexyl-D-alanylglycine but using D-phenylalanine instead of glycine.

¹H NMR [(CD₃)₂SO), 400 MHz] δ 0.72-1.64 (m, 13H), 2.87 (dd, 1H), 2.88 (d, 2H), 3.00 (dd, 1H), 3.66-3.79 (m, 2H), 4.07-4.13 (m, 1H), 4.18-4.25 (m, 1H), 4.23 (d, 1H), 4.49 (s, 2H), 4.68 (t, 1H), 5.03 (d, 1H), 6.95-7.35 (m, 17H), 7.66-7.75 (m, 1H), 8.05 (d, 1H), 8.21 (t, 1H).

### Example 12

### N-{[4-((2R,3R)-1-(4-fluorophenyl)-3-{[(2R or S)-2-(4-fluorophenyl)-2-hydroxyethyl]thio}-4-oxoazetidin-2-yl)phenoxy]acetyl}glycyl-N—[(R)-carboxy(phenyl)methyl]-3-cyclohexyl-D-alaninamide

The titled compound was prepared using the same procedure as that used for the synthesis of N-{[4-((2R,3R)-1-(4-fluorophenyl)-3-{[(2R or S)-2-(4-fluorophenyl)-2-hydroxyethyl]thio}-4-oxoazetidin-2-yl)phenoxy]acetyl}glycyl-3-cyclohexyl-D-alanylglycine but using (2R)-amino(phenyl)acetic acid instead of glycine.

¹H NMR [(CD₃)₂SO), 400 MHz] δ 0.75-1.65 (m, 13H), 2.89 (d, 2H), 3.70-3.82 (m, 2H), 4.23 (d, 1H), 4.32-4.43 (m, 1H), 4.49 (s, 2H), 4.68 (t, 1H), 4.90-4.97 (m, 1H), 5.04 (d, 1H), 6.95-7.35 (m, 17H), 8.13-8.37 (m, 3H).

### Example 13

### N-{[4-((2R,3R)-1-(4-fluorophenyl)-3-{[(2R or S)-2-(4-fluorophenyl)-2-hydroxyethyl]thio}-4-oxoazetidin-2-yl)phenoxy]acetyl}glycyl-3-cyclohexyl-D-alanyl-3-cyclohexyl-D-alanine

The titled compound was prepared using the same procedure as that used for the synthesis of N-{[4-((2R,3R)-1-(4-fluorophenyl)-3-{[(2R or S)-2-(4-fluorophenyl)-2-hydroxyethyl]thio}-4-oxoazetidin-2-yl)phenoxy]acetyl}glycyl-3-cyclohexyl-D-alanylglycine but using 3-cyclohexyl-D-alanine instead of glycine.

¹H NMR [(CD₃)₂SO), 400 MHz] δ 0.73-1.64 (m, 26H), 2.88 (d, 2H), 3.74 (d, 2H), 3.99-4.06 (m, 1H), 4.23 (d, 1H), 4.23-4.31 (m, 1H), 4.49 (s, 2H), 4.68 (t, 1H), 5.04 (d, 1H), 6.95-7.35 (m, 12H), 7.80 (d, 1H), 8.06 (d, 1H), 8.22-8.28 (m, 1H).

### Example 14

### N-{[4-((2R,3R)-1-(4-fluorophenyl)-3-{[(2R or S)-2-(4-fluorophenyl)-2-hydroxyethyl]thio}-4-oxoazetidin-2-yl)phenoxy]acetyl}glycyl-3-cyclohexyl-D-alanyl-D-valine

The titled compound was prepared using the same procedure as that used for the synthesis of N-{[4-((2R,3R)-1-(4-fluorophenyl)-3-{[(2R or S)-2-(4-fluorophenyl)-2-hydroxyethyl]thio}-4-oxoazetidin-2-yl)phenoxy]acetyl}glycyl-3-cyclohexyl-D-alanylglycine but using D-valine instead of glycine.

¹H NMR [(CD₃)₂SO), 400 MHz] δ 0.76-1.66 (m, 19H), 1.93-2.03 (m, 1H), 2.88 (d, 2H), 3.68-3.79 (m, 2H), 3.90-3.96 (m, 1H), 4.24 (d, 1H), 4.31-4.38 (m, 1H), 4.48 (s, 2H), 4.68 (t, 1H), 5.05 (d, 1H), 6.94-7.35 (m, 12H), 7.79 (d, 1H), 8.06 (d, 1H), 8.28 (t, 1H).

### Example 15

### N-{[4-((2R,3R)-1-(4-fluorophenyl)-3-{[(2R or S)-2-(4-fluorophenyl)-2-hydroxyethyl]thio}-4-oxoazetidin-2-yl)phenoxy]acetyl}glycyl-3-cyclohexyl-D-alanyl-L-valine

The titled compound was prepared using the same procedure as that used for the synthesis of N-{[4-((2R,3R)-1-(4-fluorophenyl)-3-{[(2R or S)-2-(4-fluorophenyl)-2-hydroxyethyl]thio}-4-oxoazetidin-2-yl)phenoxy]acetyl}glycyl-3-cyclohexyl-D-alanylglycine but using L-valine instead of glycine.

¹H NMR [(CD₃)₂SO), 400 MHz] δ 0.76-1.66 (m, 19H), 1.93-2.02 (m, 1H), 2.88 (d, 2H), 3.68-3.79 (m, 2H), 3.91-3.97 (m, 1H), 4.24 (d, 1H), 4.32-4.39 (m, 1H), 4.48 (s, 2H), 4.68 (t, 1H), 5.05 (d, 1H), 6.94-7.35 (m, 12H), 7.82 (d, 1H), 8.05 (d, 1H), 8.28 (t, 1H).

### Example 16

### N-{[4-((2R,3R)-1-(4-fluorophenyl)-3-{[(2R or S)-2-(4-fluorophenyl)-2-hydroxyethyl]thio}-4-oxoazetidin-2-yl)phenoxy]acetyl}glycyl-3-cyclohexyl-D-alanyl-D-lysine

To a stirred solution of N-{[4-((2R,3R)-1-(4-fluorophenyl)-3-{[(2R or S)-2-(4-fluorophenyl)-2-hydroxyethyl]thio}-4-oxoazetidin-2-yl)phenoxy]acetyl}glycyl-3-cyclohexyl-D-alanine (41.1 mg, 0.059 mmol) and tert-butyl N⁶-(tert-butoxycarbonyl)-D-lysinate hydrochloride (23.7 mg, 0.070 mmol) in DCM (3 ml, dry) was added N-methylmorpholine (20 μl, 0.18 mmol). TBTU (27 mg, 0.084 mmol) was added and the reaction mixture was stirred overnight. The formation of the intermediate was confirmed; M/z: 981(M+1) and 979 (M−1). The solvent was removed under reduced pressure. The residue was dissolved in formic acid (3.5 ml) and the reaction mixture was stirred at 35° C. for 2 hours. The formic acid was co-evaporated with toluene. The residue was dissolved in methanol (3 ml) and triethylamine (0.150 μl, 1.08 mmol) was added. The reaction mixture was stirred for 2 hours. Additional triethylamine (150 μl, 1.08 mmol) was added and the reaction mixture was stirred for 30 minutes. The solvent was removed under reduced pressure and the residue was purified with preparative HPLC on a C8 column. A gradient from 20 to 60% MeCN in a 0.1M NH₄OAc buffer was used as eluent. The pure fractions were collected and the MeCN was removed under reduced pressure. After lyophilisation, the title compound was obtained. H-NMR (400 MHz, DMSO-d₅): 0.68-0.89 (m, 3H) 1.01-1.32 (m, 7H), 1.32-1.66 (m, 1H), 2.60-2.71 (m, 2H), 2.86-2.92 (d, 2H), 3.62-3.72 (m, 1H), 3.72-3.85 (m, 2H), 4.13-4.22 (m, 1H), 4.23 (d, 1H), 4.50 (s, 2H), 4.68 (t, 1H), 5.04 (d, 1H), 6.96 (d, 2H), 7.03-7.16 (m, 4H), 7.17-7.25 (m, 2H), 7.27-7.37 (m, 4H), 7.41 (d, 1H), 8.17 (bd, 1H), 8.29 (b, 1H). M/z: 824 (M+1) and 822 (M−1).

### Example 17

### N-{[4-((2R,3R)-1-(4-fluorophenyl)-3-{[(2R or S)-2-(4-fluorophenyl)-2-hydroxyethyl]thio}-4-oxoazetidin-2-yl)phenoxy]acetyl}glycyl-3-cyclohexyl-D-alanyl-3-pyridin-4-yl-D-alanine

To a solution of N-{[4-((2R,3R)-1-(4-fluorophenyl)-3-{[(2R or S)-2-(4-fluorophenyl)-2-hydroxyethyl]thio}-4-oxoazetidin-2-yl)phenoxy]acetyl}glycyl-3-cyclohexyl-D-alanine (0.040 g, 0.058 mmol) in DMF (3 ml) was added 4-chlorophenol (0.008 g, 0.062 mmol), N-methylmorpholine (0.038 ml, 0.346 mmol) and TBTU (0.019 g, 0.059 mmol). The mixture was stirred for 30 min at RT before 3-pyridin-4-yl-D-alanine bis(trifluoroacetate) (prepared from N-(tert-butoxycarbonyl)-3-pyridin-4-yl-D-alanine and TFA) (0.034 g, 0.086 mmol) and lithium chloride (0.036 g, 0.849 mmol) was added. The reaction mixture was stirred for 30 h at RT and then purified by preparative HPLC using a gradient of 35-50% MeCN in a 0.1M ammonium acetate buffer as eluent. Freeze-drying of the pure fractions gave the desired product.

ES+ m/z: 844.5. ¹H NMR (DMSO, 500 MHz) δ: 0.66-1.70 (m, 13H), 2.82-2.96 (m, 3H), 3.02-3.12 (1H), 3.70-3.80 (2H, m), 4.20-4.41 (m, 3H), 4.48-4.56 (m, 2H), 4.68-4.74 (m, 1H), 5.07 (d, 1H), 5.66 (bs, 1H), 6.95-7.03 (m, 2H), 7.07-7.27 (m, 8H), 7.30-7.40 (m, 4H), 7.92-8.19 (m, 2H), 8.19-8.27 (m, 1H), 8.35-8.42 (m, 2H).

### Example 18

### N-{[4-(2R,3R)-1-(4-fluorophenyl)-3-{[(2R or S)-2-(4-fluorophenyl)-2-hydroxyethyl]thio}-4-oxoazetidin-2-yl)phenoxy]acetyl}glycyl-N¹-[(R)-carboxy(4-hydroxyphenyl)methyl]-3-cyclohexyl-D-alaninamide

To a solution of N-{[4-((2R,3R)-1-(4-fluorophenyl)-3-{[(2R or S)-2-(4-fluorophenyl)-2-hydroxyethyl]thio}-4-oxoazetidin-2-yl)phenoxy]acetyl}glycyl-3-cyclohexyl-D-alanine) (0.040 g, 0.058 mmol) in DMF (3 ml) was added N-methylmorpholine (0.019 ml, 0.168 mmol) and TBTU (0.019 g, 0.059 mmol). The mixture was stirred for 30 min at RT before D-4-hydroxyphenylglycine (0.012 g, 0.072 mmol) was added. The reaction mixture was stirred for 30 h at RT and then purified by preparative HPLC using a gradient of 35-50% MeCN in a 0.1M ammonium acetate buffer as eluent. Freeze-drying of the pure fractions gave the desired product.

ES+ m/z: 845.5. 1H NMR (DMSO, 400 MHz) δ: 0.69-1.72 (m, 13H), 2.86-2.98 (m, 2H), 3.70-3.85 (2H, m), 4.24-4.30 (m, 1H), 4.36-4.57 (m, 3H), 4.68-4.75 (m, 1H), 4.88-5.02 (m, 1H), 5.04-5.10 (m, 1H), 5.68 (bs, 1H), 6.61-6.72 (m, 2H), 6.94-7.03 (m, 2H), 7.05-7.28 (m, 8H), 7.30-7.41 (m, 4H), 8.02-8.13 (m, 1H), 8.20-8.38 (m, 2H), 8.23-8.45 (m, 1H). Example

### Example 19

### N-({4-[(2R,3R)-1-(4-fluorophenyl)-3-{[(2R or S)-2-(4-fluorophenyl)-2-hydroxyethyl]thio}-4-oxoazetidin-2-yl]phenoxy}acetyl)glycyl-D-lysylglycine

A mixture of N-{[4-((2R,3R)-1-(4-fluorophenyl)-3-{[(2R or S)-2-(4-fluorophenyl)-2-hydroxyethyl]thio}-4-oxoazetidin-2-yl)phenoxy]acetyl}glycyl-N6-[(9H-fluoren-9-ylmethoxy)carbonyl]-D-lysine (0.0214 g, 0.024 mmole), 3,4-dichlorophenol (0.0052 g, 0.032 mmole), N-methylmorpholin (0.007 mL, 0.060 mmole) in DMF (1 mL) was stirred, TBTU (0.0077 g, 0.024 mmole) was added. The mixture was stirred for four hours under N2-atmosphere. Glycine (0.0022 g, 0.029 mmole) was added and stirred for three days. The mixture was purified by preparative HPLC on a Kromasil C8-column using a gradient of 5-100% MeCN in 0.1M ammonium acid buffer as eluent. The solvent was removed under reduced pressure. The residue was dissolved in 1 mL of piperidine in DMF (20% by volume). After 15 minutes the solvent was removed under reduced pressure and the residue was purified by preparative HPLC on a Kromasil C8-column using a stepwise gradient of MeCN (20%, 25%, 30%, 40%, 50% and 60%) in 0.1M ammonium acid buffer as eluent. After removing the solvent under reduced pressure and freeze-drying from water, the title compound was obtained.

NMR (400 MHz, CD₃COOD) 1.37-1.51 (m, 2H), 1.57-1.78 (m, 3H), 1.82-1.94 (m, 1H), 2.82 (t, 2H), 2.92-3.05 (m, 2H), 3.72 (s, 2H), 3.82-4.02 (m, 3H), 4.43 (brt, 1H), 4.58 (s, 2H), 4.76-4.84 (m, 1H), 4.88 (d, 1H), 6.93-7.06 (m, 6H), 7.21-7.36 (m, 6H)

### Example 20

### N-({4-[(2R,3R)-1-(4-fluorophenyl)-3-{[(2R or S)-2-(4-fluorophenyl)-2-hydroxyethyl]thio}-4-oxoazetidin-2-yl]phenoxy}acetyl)glycyl-D-valylglycine

A mixture of N-({4-[(2R,3R)-1-(4-fluorophenyl)-3-{[(2R or S)-2-(4-fluorophenyl)-2-hydroxyethyl]thio}-4-oxoazetidin-2-yl]phenoxy}acetyl)glycyl-D-valine (0.020 g, 0.031 mmole), 3,4-dichlorophenol (0.0078 g, 0.048 mmole), N-methylmorpholin (0.009 mL, 0.078 mmole) in DMF (1 mL) was stirred, TBTU (0.012 g, 0.037 mmole) was added. The mixture was stirred for three hours under N2-atmosphere. Glycine (0.0028 g, 0.037 mmole) and LiCl (0.0198 g, 0.468 mmole) was added and stirred for 16 hours. The mixture was purified by preparative HPLC on a Kromasil C8-column using a gradient of 20-100% MeCN in 0.1M ammonium acid buffer as eluent. The solvent was removed under reduced pressure. After removing the solvent under reduced pressure and freeze-drying from water, the title compound was obtained.

NMR (400 MHz, CD₃COOD) 0.91 (d, 3H), 0.95 (d, 3H), 2.05-2.18 (m, 1H), 2.91-3.05 (m, 2H), 3.82 (s, 2H), 3.96-4.01 (m, 3H), 4.27 (d, 1H), 4.57 (s, 2H), 4.76-4.83 (m, 1H), 4.87 (d, 1H), 6.93-7.06 (m, 6H), 7.22-7.36 (m, 6H)

### Examples 21-43

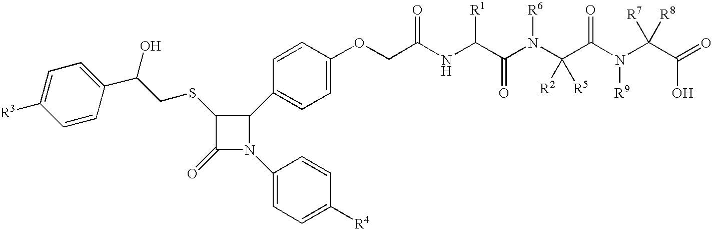

| Ex. | R2 | R5 | R7 |
|---|---|---|---|
| 21 | CH₂C₆H₅ | H | H |
| 22 | CH₂C₆H₅-p-OH | H | H |
| 23 | CH₂C₆H₅-p-CN | H | H |
| 24 | cyclohexyl | H | H |
| 25 | CH₂CH₂CH₂NH₂ | H | H |
| 26 | C(CH₃)₂C₆H₅ | H | H |
| 27 | CH₂CH(CH₃)₂ | H | H |
| 28 | CH(CH₃)₂ | CH₃ | H |
| 29 | CH₂CH₂CH₂CH₂NH(CH₃)₂ | H | H |
| 30 | CH₂SC(CH₃)₃ | H | H |
| 31 | CH₂C₆H₅ | H | C₆H₅ |
| 32 | CH₂C₆H₅-p-OH | H | C₆H₅ |
| 33 | CH₂C₆H₅-p-CN | H | C₆H₅ |
| 34 | cyclohexyl | H | C₆H₅ |
| 35 | CH₂CH₂CH₂NH₂ | H | C₆H₅ |
| 36 | CH₂CH₂CH₂CH₂NH₂ | H | C₆H₅ |
| 37 | C(CH₃)₂C₆H₅ | H | C₆H₅ |
| 38 | CH(CH₃)₂ | H | C₆H₅ |
| 39 | CH₂CH(CH₃)₂ | H | C₆H₅ |
| 40 | C(CH₃)₃ | H | C₆H₅ |
| 41 | CH(CH₃)₂ | CH₃ | C₆H₅ |
| 42 | CH₂CH₂CH₂CH₂NH(CH₃)₂ | H | C₆H₅ |
| 43 | CH₂SC(CH₃)₃ | H | C₆H₅ |

### Preparation of Starting Materials for the Above Examples

### 1-(4-Fluorophenyl)-3-(R)-[2-(4-fluorophenyl)-2-hydroxyethylthio]-4-(R)-{4-[N-(α-(R)-{N-[2-(hydroxy)-1-(S)-(carboxy)ethyl]carbamoyl}benzyl)carbamoylmethoxy]phenyl}azetidin-2-one

To a solution of 1-(4-fluorophenyl)-3-(R)-[(4-fluorobenzoyl)methylthio]-4-(R)-{4-[N-(α-(R)-{N-[2-(hydroxy)-1-(S)-(carboxy)ethyl]carbamoyl}benzyl)carbamoylmethoxy]phenyl}azetidin-2-one (Method 10; 0.039 g, 0.055 mmol) in MeOH (3 ml) was added NaBH₄ (0.005 g, 0.135 mmol). After 10 min, water (2 ml) and acetic acid (2 drops) were added before most of the solvent was removed under reduced pressure. The residue was purified by preparative HPLC using a gradient of 20-60% MeCN in 0.1M ammonium acetate buffer as eluent. After freeze-drying the desired product was obtained. NMR (DMSO, 500 MHz): 2.90-3.00 (m, 2H), 3.50 (dd, 1H), 3.60 (dd, 1H), 4.15-4.30 (m, 2H), 4.60 (ABq, 2H), 4.70-4.80 (m, 1H), 5.00-5.05 (m, 1H), 5.65 (d, 1H), 6.95-7.45 (m, 17H), 8.30-8.45 (m, 2H); m/z: 706.4.

### N-{[4-((2R,3R)-1-(4-Fluorophenyl)-3-{[2-(4-fluorophenyl)-2-oxoethyl]thio}-4-oxoazetidin-2-yl)phenoxy]acetyl}glycylglycine

A solution of [4-((2R,3R)-1-(4-fluorophenyl)-3-{[2-(4-fluorophenyl)-2-oxoethyl]thio}-4-oxoazetidin-2-yl)phenoxy]acetic acid (0.200 g, 0.414 mmol), glycylglycine methyl ester hydrochloride (0.090 g, 0.493 mmol) and N-methylmorpholine (0.150 ml) in DCM (5 ml) was stirred for 10 min. TBTU (0.170 g) was added and the mixture was stirred for 20 h. The formation of the ester was confirmed. M/z: 612.0. The solvent was removed under reduced pressure. The residue was dissolved in a mixture of MeOH (5 ml), water (1 ml) and Et₃N (0.5 ml). The solution was stirred at 50° C. for 18 h. DBN (0.050 ml, 0.405 mmol) was added and the mixture was stirred for 2 h at 50° C. Ammonium acetate buffer (0.1M, 3 ml) was added and the mixture was concentrated. The residue was purified by preparative HPLC, using a gradient of 20-50% MeCN in 0.1M ammonium acetate buffer as eluent. After freeze-drying, the title product was obtained. M/z: 598.2. 1H NMR. (DMSO, 400 MHz): 3.50 (d, 2H), 3.75 (d, 2H), 4.32 (d, 1H), 4.35 (ABq, 2H), 4.46-4.53 (m, 2H), 5.15 (d, 1H), 6.94-7.00 (m, 2H), 7.10-7.25 (m, 4H), 7.29-7.39 (m, 4H), 7.68-7.81 (m, 1H), 7.98-8.04 (m, 2H), 8.30-8.36 (m, 1H).

### 1-(4-Fluorophenyl)-3-(R)-[2-(4-fluorophenyl)-2-hydroxyethylthio]-4-(R)-{4-[N-{N-[2-(hydroxy)-1-(R)-(carboxy)ethyl]carbamoylmethyl}carbamoylmethoxy]phenyl}azetidin-2-one

To a solution of 1-(4-fluorophenyl)-3-(R)-[(4-fluorobenzoyl)methylthio]-4-(R)-{4-[N-{N-[2-(hydroxy)-1-(R)-(carboxy)ethyl]-carbamoylmethyl}carbamoylmethoxy]phenyl} azetidin-2-one (Method 13; 0.028 g, 0.045 mmol) in MeOH (3 ml) was added NaBH₄ (0.010 g, 0.264 mmol). After 10 minutes the solvent was removed under reduced pressure and the residue was purified by preparative HPLC using a gradient of 20-50% MeCN in 0.1M ammonium acetate buffer as eluent. After freeze-drying the desired product was obtained in 0.014 g (50%). NMR (CD₃COOD, 400 MHz): 3.00-3.20 (m, 2H), 3.95 (dd, 1H), 4.00-4.15 (m, 2H), 4.25 (ABq, 2H), 4.70 (s, 2H), 4.70-4.80 (m, 1H), 4.85-5.00 (m, 2H), 6.95-7.10 (m, 6H), 7.25-7.45 (m, 6H); m/z: 630.1.

### 1-(4-Fluorophenyl)-3-(R)-[2-(4-fluorophenyl)-2-hydroxyethylthio]-4-(R)-{4-[N-{N-[2-(phenyl)-1-(R)-(carboxy)ethyl]carbamoylmethyl}carbamoylmethoxy]phenyl}azetidin-2-one

1-(4-Fluorophenyl)-3-(R)-[(4-fluorobenzoyl)methylthio]-4-(R)-{4-[N—{N-[2-(phenyl)-1-(R)-(carboxy)ethyl]carbamoylmethyl}carbamoylmethoxy]phenyl}azetidin-2-one (15 mg, 0.022 mmol) was dissolved in methanol (1 ml) and sodium borohydride (4 mg) was added. The solvent was evaporated and the residue was purified by preparative HPLC using a gradient from 10% to 100% MeCN in 0.1 M ammonium acetate buffer as mobile phase. Lyophilisation of the product fraction gave the desired product. NMR (400 MHz, CD₃COOD): 3.02-3.17 (m, 3H), 3.19-3.25 (m, 1H), 4.06-4.17 (m, 3H), 4.66 (s, 2H), 4.87-4.96 (m, 3H), 6.97-7.05 (m, 6H), 7.10-7.40 (m, 12H); m/z 688.3 (m-H).

### 4-chlorophenyl {4-[(2R,3R)-1-(4-fluorophenyl)-3-{[(2R or S)-2-(4-fluorophenyl)-2-hydroxyethyl]thio}-4-oxoazetidin-2-yl]phenoxy}acetate

A mixture of {4-[(2R,3R)-1-(4-fluorophenyl)-3-{[(2R or S)-2-(4-fluorophenyl)-2-hydroxyethyl]thio}-4-oxoazetidin-2-yl]phenoxy}acetic acid (2.81 g, 5.79 mmole), N-methylmorpholin (1.91 mL, 17.36 mmole) and TBTU (2.26 g, 7.04 mmole) in dichloromethane (30 mL) was stirred for 5 minutes. 4-chlorophenol (0.762 g, 5.93 mmole) was added. The mixture was stirred for 16 hours. The solvent was evaporated under reduced pressure. The mixture was purified by preparative HPLC on a Kromasil C8-column using a gradient of 20-95% MeCN in 0.1M ammonium acid buffer as eluent. The solvent was removed under reduced pressure. After removing the solvent under reduced pressure and freeze-drying from water, the title compound was obtained.

NMR (400 MHz, CD₃COOD) 2.92-3.05 (m, 2H), 3.98 (d, 1H), 4.72-4.82 (m, 1H), 4.88 (d, 1H), 4.99 (s, 2H), 6.92-7.06 (m, 6H), 7.12 (d, 2H), 7.22-7.39 (m, 8H)

### N-({4-[(2R,3R)-1-(4-fluorophenyl)-3-{[(2R or S)-2-(4-fluorophenyl)-2-hydroxyethyl]thio}-4-oxoazetidin-2-yl]phenoxy}acetyl)glycyl-D-valine

A mixture of 4-chlorophenyl 4-[(2R,3R)-1-(4-fluorophenyl)-3-{[(2R or S)-2-(4-fluorophenyl)-2-hydroxyethyl]thio)-4-oxoazetidin-2-yl]phenoxy}acetate (0.095 g, 0.159 mmole), glycyl-D-valine hydrochloride (0.037 g, 0.176 mmole), lithium chloride (0.109 g, 2.57 mmole) and N-methylmorpholin (0.037 mL, 0.336 mmole) in DMF (1 mL) was stirred under N2-atmosphere. After one hour N-methylmorpholine (0.018 mL, 0.159 mmole) was added. After one day DMF (1 mL) was added and lithium chloride (one small spatula, about 0.02 g). After two days glycyl-D-valine hydrochloride (0.010-0.015 g) in DMF (1 mL) and lithium chloride (one small spatula, about 0.02 g) was added. After three days the solvent was evaporated under reduced pressure. The mixture was purified by preparative HPLC on a Kromasil C8-column using a gradient of 20-100% MeCN in 0.1M ammonium acid buffer as eluent. The solvent was removed under reduced pressure. KHSO₄-solution (0.3M) was added and the water layer was extracted with EtOAc. The organic layer was washed with brine, dried, filtered and evaporated under reduced pressure to give the title compound.

NMR (500 MHz, CD₃COOD) 0.93-0.99 (m, 6H), 2.13-2.23 (m, 1H), 2.94-3.08 (m, 2H), 3.96-4.09 (m, 3H), 4.34-4.39 (m, 1H), 4.60 (s, 2H), 4.79-4.86 (m, 1H), 4.91 (d, 1H), 6.96-7.04 (m, 4H), 7.06 (d, 2H), 7.26-7.38 (m, 6H)

It will be appreciated by those skilled in the art that the examples may be modified within the realms of the invention, why the invention is not limited to particular embodiments.

### Absorption

Absorption of the compounds of formula (I) was tested in a Caco-2 cells model (Gastroenterology 1989, 96, 736):

| | Caco value |
|---|---|
| Compound (I) | (10⁻⁶ cm/sec) |
| N-{[4-((2R,3R)-1-(4-fluorophenyl)-3-{[(2R or S)- | 0.12 |
| 2-(4-fluorophenyl)-2-hydroxyethyl]thio}-4- | |
| oxoazetidin-2-yl)phenoxy]acetyl}glycyl-3- | |
| methyl-D-valylglycine | |

1. A compound of formula (I): 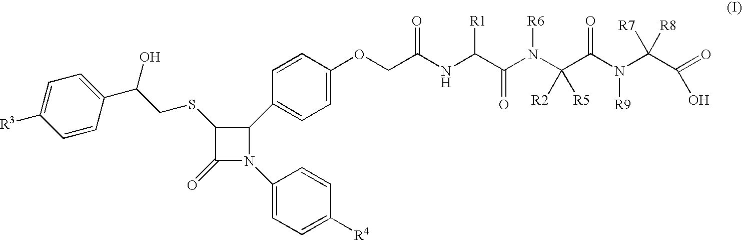
wherein:
R¹ is hydrogen, C₁₋₆alkyl, C₃₋₆cycloalkyl or aryl;
R², R⁵, R⁷ and R⁸ are independently hydrogen, a branched or unbranched C₁₋₆alkyl, C₃₋₆cycloalkyl or aryl; wherein said C₁₋₆alkyl may be optionally substituted by one or more hydroxy, amino, guanidino, cyano, carbamoyl, carboxy, C₁₋₆alkoxy, aryl C₁₋₆alkoxy, (C₁-C₄alkyl)₃Si, N—(C₁₋₆alkyl)amino, N,N—(C₁₋₆alkyl)₂-amino, C₁₋₆alkylS(O)ₐ, C₃₋₆cycloalkyl, aryl or aryl C₁₋₆ alkylS(O)ₐ, wherein a is 0-2; and wherein any aryl group may be optionally substituted by one or two substituents selected from halo, hydroxy, C₁₋₆alkyl, C₁₋₆alkoxy, and cyano;
R³ is hydrogen, alkyl, halo, C₁₋₆alkoxy or C₁₋₆ alkylS—;
R⁴ is hydrogen, C₁₋₆ alkyl, halo or C₁₋₆alkoxy; and
R⁶ and R⁹ are, independently, hydrogen, C₁₋₆ alkyl, or arylC₁₋₆ alkyl;
wherein R⁵ and R² may form a ring with 2-7 carbon atoms and wherein R⁶ and R² may form a ring with 3-6 carbon atoms;
or a pharmaceutically acceptable salt, solvate, solvate of such a salt or a prodrug thereof

2. A compound of formula (I2): 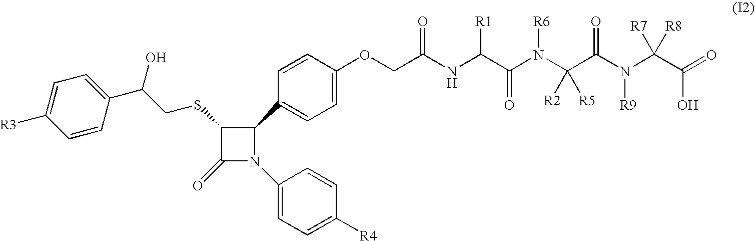
wherein:
R¹ is hydrogen, C₁₋₆alkyl, C₃₋₆cycloalkyl or aryl;
R², R⁵, R⁷ and R⁸ are independently hydrogen, a branched or unbranched C₁₋₆alkyl, hydroxy, amino, guanidino, cyano, carbamoyl, carboxy, C₁₋₆alkoxy, aryl C₁₋₆alkoxy, (C₁-C₄alkyl)₃Si, N—(C₁₋₆alkyl)amino, N,N—(C₁₋₆alkyl)₂amino, C₁₋₆alkylS(O)ₐ, C₃₋₆cycloalkyl, aryl or aryl C₁₋₆ alkylS(O)ₐ, wherein a is 0-2; and wherein any aryl group may be optionally substituted by one or two substituents selected from halo, hydroxy, C₁₋₆alkyl, C₁₋₆alkoxy, and cyano;
R³ is hydrogen, alkyl, halo, C₁₋₆alkoxy or C₁₋₆ alkylS—;
R⁴ is hydrogen, C₁₋₆ alkyl, halo or C₁₋₆alkoxy;
R⁶ and R⁹ are, independently, hydrogen, C₁₋₆ alkyl, and arylC₁₋₆ alkyl;
wherein R⁵ and R² may form a ring with 2-7 carbon atoms and wherein R⁶ and R² may form a ring with 3-6 carbon atoms;
or a pharmaceutically acceptable salt, solvate, solvate of such a salt or a prodrug thereof.

3. A compound according to claim 1, wherein: R¹ is hydrogen.

4. A compound according to claim 1, wherein: R² and R⁵ are, independently, hydrogen or aryl.

5. A compound according to claim 1, wherein: R³ is halo.

6. A compound according to claim 1, wherein: R³ is fluorine.

7. A compound according to claim 1, wherein: R⁴ is halo.

8. A compound according to claim 1, wherein: R⁴ is fluorine.

9. A compound according to claim 1, wherein: R⁶ is hydrogen.

10. A compound according to claim 1, wherein: R⁷ and R⁸ are, independently, hydrogen or a branched or unbranched C₁₋₆alkyl.

11. A compound according to claim 1 wherein:
R¹ is hydrogen;
R², R⁵, R⁷ and R⁸ are independently hydrogen, a branched or unbranched C₁₋₆alkyl, C₃₋₆cycloalkyl or aryl; wherein said C₁₋₆alkyl may be optionally substituted by one or more hydroxy, amino, carbamoyl, carboxy, C₁₋₆alkoxy, aryl C₁₋₆alkoxy, (C₁-C₄alkyl)₃Si, N—(C₁₋₆alkyl)amino, N,N—(C₁₋₆alkyl)₂amino, C₃₋₆cycloalkyl, or aryl; and wherein any aryl group may be optionally substituted by one or two substituents selected from halo, hydroxy, C₁₋₆alkyl, C₁₋₆alkoxy, and cyano;
R³ is hydrogen, alkyl, halo or C₁₋₆alkoxy;
R⁴ is hydrogen, C₁₋₆ alkyl, halo or C₁₋₆alkoxy; and
R⁶ is hydrogen and R⁹ is hydrogen, C₁₋₆ alkyl, or arylC₁₋₆ alkyl.

12. One or more compounds selected from:
N-{(2R)-2-[(N-{[4-((2R,3R)-1-(4-fluorophenyl)-3-{[2-(4-fluorophenyl)-2-hydroxyethyl]thio}-4-oxoazetidin-2-yl)phenoxy]acetyl}glycyl)amino]-2-phenylacetyl}glycine;
N-{[4-((2R,3R)-1-(4-fluorophenyl)-3-{[2-(4-fluorophenyl)-2-hydroxyethyl]thio}-4-oxoazetidin-2-yl)phenoxy]acetyl}glycylglycyl-N-benzylglycine;
N-{[4-((2R,3R)-1-(4-fluorophenyl)-3-{[2-(4-fluorophenyl)-2-hydroxyethyl]thio}-4-oxoazetidin-2-yl)phenoxy]acetyl}glycylglycyl-N-ethylglycine;
N-{[4-((2R,3R)-1-(4-fluorophenyl)-3-{[2-(4-fluorophenyl)-2-hydroxyethyl]thio}-4-oxoazetidin-2-yl)phenoxy]acetyl}glycylglycyl-3-methyl-D-valine.hydrogen;
N-{[4-((2R,3R)-1-(4-fluorophenyl)-3-{[(2R or S)-2-(4-fluorophenyl)-2-hydroxyethyl]thio}-4-oxoazetidin-2-yl)phenoxy]acetyl}glycyl-3-methyl-D-valylglycine;
N-{[4-((2R,3R)-1-(4-fluorophenyl)-3-{[(2R)-2-(4-fluorophenyl)-2-hydroxyethyl]thio}-4-oxoazetidin-2-yl)phenoxy]acetyl}glycyl-3-methyl-D-valyl-D-serine;
N-{[4-((2R,3R)-1-(4-fluorophenyl)-3-{[(2R)-2-(4-fluorophenyl)-2-hydroxyethyl]thio}-4-oxoazetidin-2-yl)phenoxy]acetyl}glycyl-3-cyclohexyl-D-alanylglycine;
N-{[4-((2R,3R)-1-(4-fluorophenyl)-3-{[(2R)-2-(4-fluorophenyl)-2-hydroxyethyl]thio}-4-oxoazetidin-2-yl)phenoxy]acetyl}glycyl-3-cyclohexyl-D-alanylglycine;
N-{[4-((2R,3R)-1-(4-fluorophenyl)-3-{[(2R)-2-(4-fluorophenyl)-2-hydroxyethyl]thio}-4-oxoazetidin-2-yl)phenoxy]acetyl}glycyl-3-cyclohexyl-D-alanyl-D-alanine;
N-{[4-((2R,3R)-1-(4-fluorophenyl)-3-{[(2R)-2-(4-fluorophenyl)-2-hydroxyethyl]thio}-4-oxoazetidin-2-yl)phenoxy]acetyl}glycyl-N—[(R)-carboxy(phenyl)methyl]-3-cyclohexyl-D-alaninamide;
N-{[4-((2R,3R)-1-(4-fluorophenyl)-3-{[(2R)-2-(4-fluorophenyl)-2-hydroxyethyl]thio}-4-oxoazetidin-2-yl)phenoxy]acetyl}glycyl-3-cyclohexyl-D-alanyl-D-valine; and
N-{[4-((2R,3R)-1-(4-fluorophenyl)-3-{[(2R)-2-(4-fluorophenyl)-2-hydroxyethyl]thio}-4-oxoazetidin-2-yl)phenoxy]acetyl}glycyl-3-cyclohexyl-D-alanyl-D-lysine.

13. A method of treating or preventing a hyperlipidemic condition comprising the administration of an effective amount of a compound according to claim 1 to a mammal in need thereof.

14. A method of treating or preventing atherosclerosis comprising the administration of an effective amount of a compound according to claim 1 to a mammal in need thereof.

15. A method for treating or preventing Alzheimers' disease comprising the administration of an effective amount of a compound according to claim 1 to a mammal in need thereof.

16. A method for treating or preventing cholesterol associated tumor comprising the administration of an effective amount of a compound according to claim 1 to a mammal in need thereof.

17. A pharmaceutical formulation comprising a compound according to claim 1 in admixture with a pharmaceutically acceptable adjuvant, diluent and/or carrier.

18. A combination of a compound according to formula (I) or (I2) 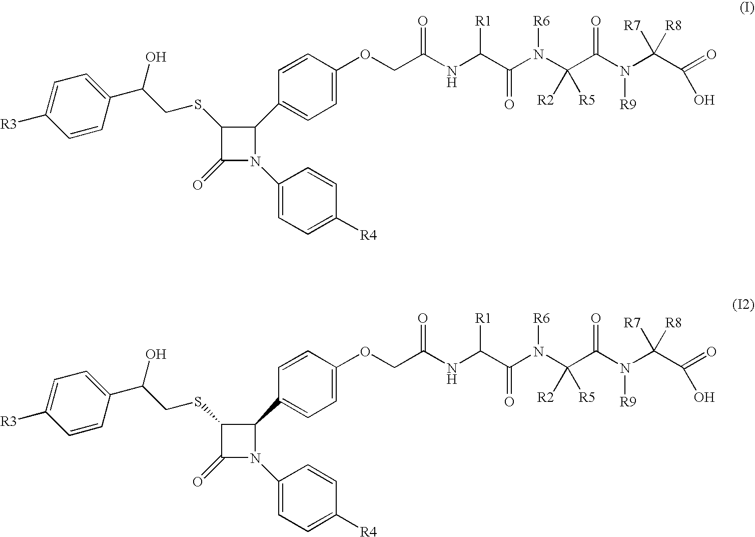
wherein:
R¹ is hydrogen, C₁₋₆alkyl, C₃₋₆cycloalkyl or aryl;
R², R⁵, R⁷ and R⁸ are independently hydrogen, a branched or unbranched C₁₋₆alkyl, C₃₋₆cycloalkyl or aryl; wherein said C₁₋₆alkyl may be optionally substituted by one or more hydroxy, amino, guanidino, cyano, carbamoyl, carboxy, C₁₋₆alkoxy, aryl C₁₋₆alkoxy, (C₁-C₄alkyl)₃Si, N—(C₁₋₆alkyl)amino, N,N—(C₁₋₆alkyl)₂amino, C₁₋₆alkylS(O)ₐ, C₃₋₆ cycloalkyl, aryl or aryl C₁₋₆ alkylS(O)ₐ, wherein a is 0-2; and wherein any aryl group may be optionally substituted by one or two substituents selected from halo, hydroxy, C₁₋₆alkyl, C₁₋₆alkoxy, and cyano;
R³ is hydrogen, alkyl, halo, C₁₋₆alkoxy or C₁₋₆ alkylS—;
R⁴ is hydrogen, C₁₋₆ alkyl, halo or C₁₋₆alkoxy; and
R⁶ and R⁹ are, independently, hydrogen, C₁₋₆ alkyl, or arylC₁₋₆ alkyl;
wherein R⁵ and R² may form a ring with 2-7 carbon atoms and wherein R⁶ and R² may form a ring with 3-6 carbon atoms;
or a pharmaceutically acceptable salt, solvate, solvate of such a salt or a prodrug thereof;
with a PPAR alpha and/or gamma agonist.

19. A combination of a compound according to formula (I) or (I2) 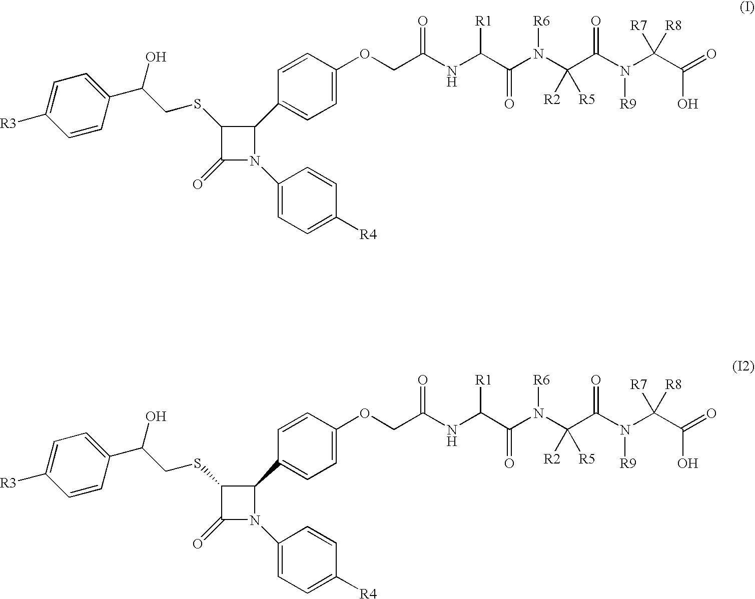
wherein:
R¹ is hydrogen, C₁₋₆alkyl, C₃₋₆cycloalkyl or aryl;
R², R⁵, R⁷ and R⁸ are independently hydrogen, a branched or unbranched C₁₋₆alkyl, C₃₋₆ cycloalkyl or aryl; wherein said C₁₋₆alkyl may be optionally substituted by one or more hydroxy, amino, guanidino, cyano, carbamoyl, carboxy, C₁₋₆alkoxy, aryl C₁₋₆alkoxy, (C₁-C₄alkyl)₃Si, N—(C₁₋₆alkyl)amino, N,N—(C₁₋₆alkyl)₂amino, C₁₋₆alkylS(O)ₐ, C₃₋₆ cycloalkyl, aryl or aryl C₁₋₆ alkylS(O)ₐ, wherein a is 0-2; and wherein any aryl group may be optionally substituted by one or two substituents selected from halo, hydroxy, C₁₋₆alkyl, C₁₋₆alkoxy, and cyano;
R³ is hydrogen, alkyl, halo, C₁₋₆alkoxy or C₁₋₆ alkylS—;
R⁴ is hydrogen, C₁₋₆ alkyl, halo or C₁₋₆alkoxy; and
R⁶ and R⁹ are, independently, hydrogen, C₁₋₆ alkyl, or arylC₁₋₆ alkyl;
wherein R⁵ and R² may form a ring with 2-7 carbon atoms and wherein R⁶ and R² may form a ring with 3-6 carbon atoms;
or a pharmaceutically acceptable salt, solvate, solvate of such a salt or a prodrug thereof;
with an HMG Co-A reductase inhibitor.

20. A process for preparing a compound of formula (I) 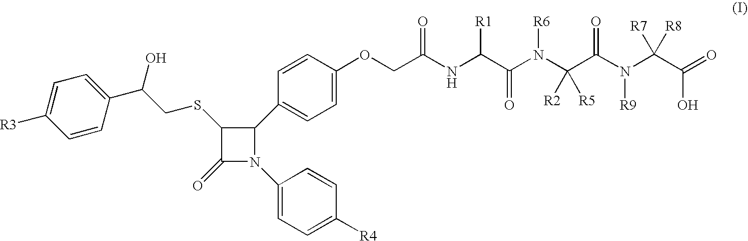
wherein:
R¹ is hydrogen, C₁₋₆alkyl, C₃₋₆cycloalkyl or aryl;
R², R⁵, R⁷ and R⁸ are independently hydrogen, a branched or unbranched C₁₋₆alkyl, C₃₋₆cycloalkyl or aryl; wherein said C₁₋₆alkyl may be optionally substituted by one or more hydroxy, amino, guanidino, cyano, carbamoyl, carboxy, C₁₋₆alkoxy, aryl C₁₋₆alkoxy, (C₁-C₄alkyl)₃Si, N—(C₁₋₆alkyl)amino, N,N—(C₁₋₆alkyl)₂amino, C₁₋₆alkylS(O)ₐ, C₃₋₆cycloalkyl, aryl or aryl C₁₋₆ alkylS(O)ₐ, wherein a is 0-2; and wherein any aryl group may be optionally substituted by one or two substituents selected from halo, hydroxy, C₁₋₆alkyl, C₁₋₆alkoxy, and cyano;
R³ is hydrogen, alkyl, halo, C₁₋₆alkoxy or C₁₋₆ alkylS—;
R⁴ is hydrogen, C₁₋₆ alkyl, halo or C₁₋₆alkoxy; and
R⁶ and R⁹ are, independently, hydrogen, C₁₋₆ alkyl, or arylC₁₋₆ alkyl;
wherein R⁵ and R² may form a ring with 2-7 carbon atoms and wherein R⁶ and R² may form a ring with 3-6 carbon atoms;
or a pharmaceutically acceptable salt, solvate, solvate of such a salt or a prodrug thereof which process (wherein variable groups are, unless otherwise specified, as defined in formula (I)) comprising of any of the steps:
Process 1) reacting a compound of formula (II): 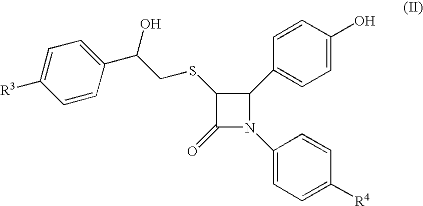
with a compound of formula (III): 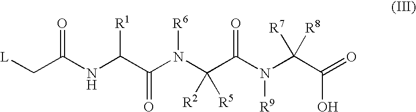
wherein L is a displaceable group; or
Process 2) reacting an acid of formula (IV): 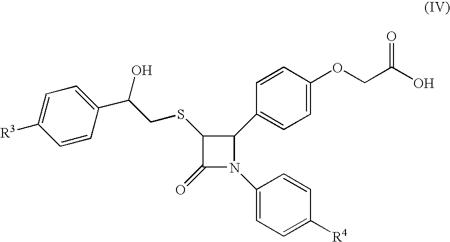
or an activated derivative thereof; with an amine of formula (V): 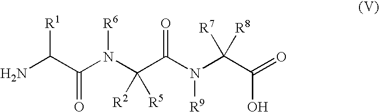
or
Process 3): reacting an acid of formula (VI): 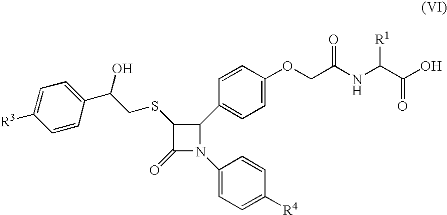
or an activated derivative thereof, with an amine of formula (VII): 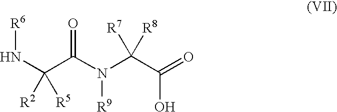
or
Process 4): reducing a compound of formula (VIII): 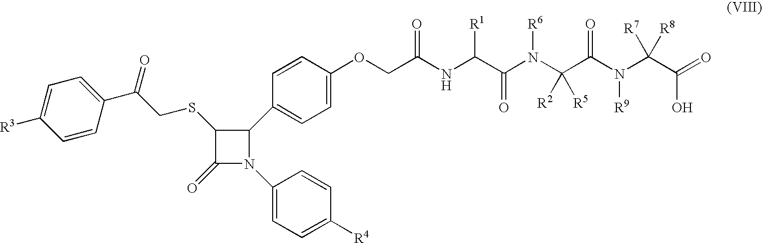
or
Process 5): reacting a compound of formula (IX): 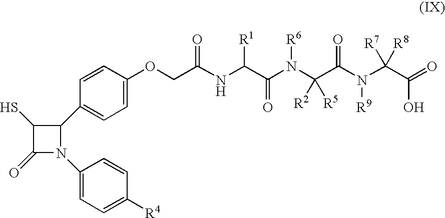
with a compound of formula (X): 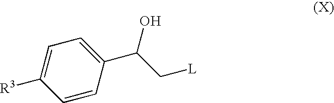
wherein L is a displaceable group; or
Process 6): reacting a compound of formula (XI): 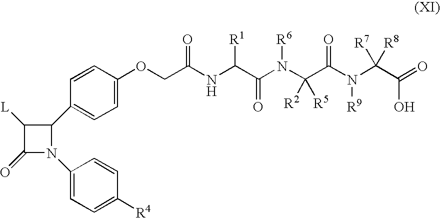
wherein L is a displaceable group; with a compound of formula (XII): 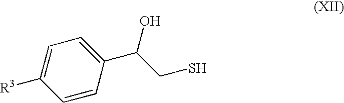
or
Process 7): De-esterifying a compound of formula (XIII) 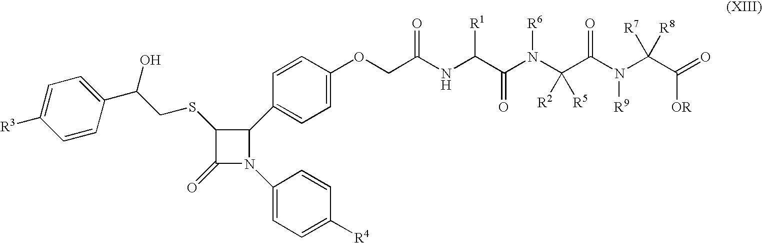
wherein the group C(O)OR is an ester group.

21. A compound according to claim 2, wherein: R¹ is hydrogen.

22. A compound according to claim 2, wherein: R² and R⁵ are, independently, hydrogen or aryl.

23. A compound according to claim 2, wherein: R³ is halo.

24. A compound according to claim 2, wherein: R³ is fluorine.

25. A compound according to claim 2, wherein: R⁴ is halo.

26. A compound according to claim 2, wherein: R⁴ is fluorine.

27. A compound according to claim 1, wherein: R⁶ is hydrogen.

28. A compound according to claim 2, wherein: R⁷ and R⁸ are, independently, hydrogen or a branched or unbranched C₁₋₆alkyl.

29. A compound according to claim 2 wherein:
R¹ is hydrogen;
R², R⁵, R⁷ and R⁸ are independently hydrogen, a branched or unbranched C₁₋₆alkyl, C₃₋₆cycloalkyl or aryl; wherein said C₁₋₆alkyl may be optionally substituted by one or more hydroxy, amino, carbamoyl, carboxy, C₁₋₆alkoxy, aryl C₁₋₆alkoxy, (C₁-C₄alkyl)₃Si, N—(C₁₋₆alkyl)amino, N,N—(C₁₋₆alkyl)₂amino, C₃₋₆cycloalkyl, or aryl; and wherein any aryl group may be optionally substituted by one or two substituents selected from halo, hydroxy, C₁₋₆alkyl, C₁₋₆alkoxy, and cyano;
R³ is hydrogen, alkyl, halo or C₁₋₆alkoxy;
R⁴ is hydrogen, C₁₋₆ alkyl, halo or C₁₋₆alkoxy; and
R⁶ is hydrogen and R⁹ is hydrogen, C₁₋₆ alkyl, or arylC₁₋₆ alkyl.

30. A method of treating or preventing a hyperlipidemic condition comprising the administration of an effective amount of a compound according to claim 2 to a mammal in need thereof.

31. A method of treating or preventing atherosclerosis comprising the administration of an effective amount of a compound according to claim 2 to a mammal in need thereof.

32. A method for treating or preventing Alzheimers' disease comprising the administration of an effective amount of a compound according to claim 2 to a mammal in need thereof.

33. A method for treating or preventing a cholesterol associated tumor comprising the administration of an effective amount of a compound according to claim 2 to a mammal in need thereof.

34. A pharmaceutical formulation comprising a compound according to claim 2 in admixture with a pharmaceutically acceptable adjuvant, diluent and/or carrier.
